(19) United States
(12) Patent Application Publication
(10) Pub. No.: US 2024/0315237 A1
(43) Pub. Date: Sep. 26, 2024

(54) METHODS FOR SINGLE CELL ANALYSIS OF GLUCOSE INTERNALIZATION

(71) Applicant: ABBOTT DIABETES CARE INC., Alameda, CA (US)

(72) Inventors: Joel D. PAPROCKI, Saint Francis, WI (US); Patrick J. MACDONALD, Mundelein, IL (US)

(73) Assignee: ABBOTT DIABETES CARE INC., Alameda, CA (US)

(21) Appl. No.: 18/582,219

(22) Filed: Feb. 20, 2024

Related U.S. Application Data
(60) Provisional application No. 63/601,516, filed on Nov. 21, 2023.
(60) Provisional application No. 63/446,788, filed on Feb. 17, 2023.

(51) Int. Cl. A01N 1/02; G01N 33/49; G01N 33/557

(52) U.S. Cl. CPC A01N 1/0226; G01N 33/49; G01N 33/557

(57) ABSTRACT

The subject matter disclosed herein relates to methods for analyzing and determining the kinetics of glucose internalization in single red blood cells, e.g., obtained from a patient. The present disclosure further provides kits and systems for performing the methods disclosed herein.

### CROSS-REFERENCE TO RELATED APPLICATIONS

The application claims priority to U.S. Provisional Application No. 63/446,788, filed Feb. 17, 2023, and U.S. Provisional Application No. 63/601,516, filed Nov. 21, 2023, the contents of each of which are incorporated herein by reference herein in their entireties.

### FIELD

The subject matter disclosed herein relates to methods for determining the kinetics of glucose internalization in single red blood cells, e.g., obtained from a patient.

### BACKGROUND

The measurement of various analytes within an individual can sometimes be vital for monitoring the condition of their health. During normal circulation of red blood cells in a mammal such as a human body, glucose molecules attach to hemoglobin, which is referred to as glycosylated hemoglobin (also referred to as glycated hemoglobin). The higher the amount of glucose in the blood, the higher the percentage of circulating hemoglobin molecules with glucose molecules attached. Since glucose molecules stay attached to hemoglobin for the life of the red blood cells, the level of glycosylated hemoglobin reflects an average blood glucose level over that period.

Most of hemoglobin is a type called HbA. When glucose molecules attach to HbA molecules, glycosylated HbA is formed, which is referred to as HbA1. HbA1 has three components: HbA1a, HbA1b and HbA1c. Because glucose has the highest affinity with N-terminal amino group of the hemoglobin beta chain, leading to HbA1c, a measure of HbA1c in blood (HbA1c test) is often used as an indication of a subject's average blood glucose level over a 120 day period (the average lifespan of a red blood cell). The HbA1c test is performed by drawing a blood sample from a subject at a medical professional's office, which is then analyzed in a laboratory. The HbA1c test can be used as a screening and diagnostic test for pre-diabetes and diabetes. A subject's glucose exposure as determined by HbA1c levels is one of the primary factors used in making diagnosis and/or therapy decisions. A subject's laboratory HbA1c level (also referred to in the art as a measured HbA1c) is compared to a normal or health range when diagnosing and/or treating the subject.

However, while HbA1c continues to be the benchmark biomarker for glycemic management, HbA1c levels can vary based on factors other than glycemia. For example, one such factor that can affect HbA1c levels is the uptake and consumption rate of glucose by red blood cells. Therefore, the diagnoses and glycemic management treatments are sometimes based on an incorrect glucose exposure. Accordingly, there is a need in the art for improved methods of determining glucose uptake in red blood cells to allow for a more personalized approach to the diagnosis or treatment of pre-diabetic or diabetic patients.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

The present disclosure provides methods for analyzing glucose internalization in single red blood cells. In certain embodiments, the method includes (a) anchoring a population of red blood cells to a substrate to generate a population of anchored red blood cells, (b) contacting the population of anchored red blood cells with a labeled glucose analog (e.g., a fluorescently labeled glucose analog), (c) obtaining sequential images of the population of anchored red blood cells or a subset thereof of (b) for a length of time and (d) analyzing the internalization of the labeled glucose analog (e.g., the fluorescently labeled glucose analog) in one or more single red blood cells of the population of anchored red blood cells or the subset thereof. In certain embodiments, the method includes (a) providing a population of red blood cells, (b) anchoring the population of red blood cells to a substrate to generate a population of anchored red blood cells, (c) contacting the population of anchored red blood cells with a labeled glucose analog (e.g., a fluorescently labeled glucose analog), (d) obtaining sequential images of the population of anchored red blood cells or a subset thereof of (c) for a length of time and (e) analyzing the internalization of the labeled glucose analog (e.g., the fluorescently labeled glucose analog) in each single red blood cell of the population of anchored red blood cells or the subset thereof. In certain embodiments, the methods of the present disclosure are in vitro methods.

In certain embodiments, the population of red blood cells is obtained from a patient. In certain embodiments, the population of red blood cells is obtained from a single patient. In certain embodiments, the population of red blood cells is obtained from a sample of a patient. In certain embodiments, the population of red blood cells is obtained from a sample of a single patient. In certain embodiments, the patient is a nondiabetic, has prediabetes or has diabetes.

In certain embodiments, the fluorescently labeled glucose analog is 2-NBDG (2-(N-(7-Nitrobenz-2-oxa-1,3-diazol-4-yl)Amino)-2-Deoxyglucose). In certain embodiments, the fluorescently labeled glucose analog is contacted with the population of anchored red blood cells at a concentration from about 1 mM to about 30 mM. In certain embodiments, the population of anchored red blood cells are contacted with the fluorescently labeled glucose analog at more than one concentration, at more than two concentrations, at more than three concentrations, at more than four concentrations or at more than five concentrations. In certain embodiments, the population of anchored red blood cells are contacted with the fluorescently labeled glucose analog at one concentration. In certain embodiments, the population of anchored red blood cells are contacted with the fluorescently labeled glucose analog at two different concentrations. In certain embodiments, the population of anchored red blood cells are contacted with the fluorescently labeled glucose analog at three different concentrations. In certain embodiments, the population of anchored red blood cells are contacted with the fluorescently labeled glucose analog at four different concentrations.

In certain embodiments, anchoring the population of red blood cells to the substrate includes: (a) coating the substrate with a serum albumin-biotin conjugate coupled to an avidin or an analog thereof (e.g., to generate a coated substrate), (b) contacting the population of red blood cells with an antigen-binding molecule-biotin conjugate to generate a population of red blood cells coupled to the antigen-binding molecule-biotin conjugate and (c) contacting the population of red blood cells coupled to the antigen-binding molecule-biotin conjugate with the coated substrate of (a) to generate a population of anchored red blood cells. In certain embodiments, the antigen-binding molecule-biotin conjugate binds to a protein on the surface of the red blood cells. In certain embodiments, the protein is not a GLUT1 receptor. In certain embodiments, the protein on the surface of the red blood cells is a glycophorin.

In certain embodiments, the population of red blood cells includes from about to 5 about 10,000 red blood cells.

In certain embodiments, analyzing the internalization of the fluorescently labeled glucose analog in each single red blood cell of the population of anchored red blood cells or the subset thereof comprises determining an intracellular level of the glucose analog in each red blood cell. In certain embodiments, the method further includes determining an average or median intracellular level of the glucose analog in the population of red blood cells or the subset thereof. In certain embodiments, the method further includes comparing the intracellular level of the glucose analog in each red blood cell or the average or median intracellular level of the glucose analog to a reference intracellular level (e.g., of the glucose analog). In certain embodiments, the reference intracellular level (e.g., of the glucose analog) is a value obtained from a plurality of subjects having at least one demographic metric in common with the patient. In certain embodiments, the reference intracellular level (e.g., of the glucose analog) is a value obtained from a plurality of subjects having at least two demographic metrics in common with the patient. In certain embodiments, at least one of the demographic metrics is race. The present disclosure further includes methods of providing a personalized diagnosis and/or treatment to a patient using the intracellular level of the glucose analog or the average or median intracellular level of the glucose analog.

In certain embodiments, analyzing the internalization of the fluorescently labeled glucose analog in each single red blood cell of the population of anchored red blood cells or the subset thereof comprises determining an intracellular level of the glucose analog in each red blood cell. In certain embodiments, the method further includes determining an average or median intracellular level of the glucose analog in the population of red blood cells or the subset thereof. In certain embodiments, the method further includes comparing the intracellular level of the glucose analog in each red blood cell or the average or median intracellular level of the glucose analog to a reference extracellular level. In certain embodiments, the reference extracellular level is a known or experimentally measured value of the glucose analog concentration brought into contact with the anchored cells. The present disclosure further includes methods of providing a personalized diagnosis and/or treatment to a patient using the intracellular level of the glucose analog or the average or median intracellular level of the glucose analog.

In certain embodiments, analyzing the internalization of the fluorescently labeled glucose analog in each single red blood cell of the population of anchored red blood cells or the subset thereof comprises determining a Vₘₐₓ for each red blood cell. In certain embodiments, the method further includes determining an average or median Vₘₐₓ of the population of red blood cells or the subset thereof. In certain embodiments, the method further includes comparing the Vₘₐₓ or the average or median Vₘₐₓ to a reference Vₘₐₓ. In certain embodiments, the reference Vₘₐₓ is a value obtained from a plurality of subjects having at least one demographic metric in common with the patient. In certain embodiments, the reference Vₘₐₓ is a value obtained from a plurality of subjects having at least two demographic metrics in common with the patient. In certain embodiments, at least one of the demographic metrics is race. The present disclosure further includes methods of providing a personalized diagnosis and/or treatment to a patient using the Vₘₐₓ or the average or median Vₘₐₓ. The present disclosure further includes methods of adjusting a patient's HbA1c to the Vₘₐₓ or the average or median Vₘₐₓ.

In certain embodiments, analyzing the internalization of the fluorescently labeled glucose analog in each single red blood cell of the population of anchored red blood cells or the subset thereof comprises determining a ratio of the intracellular level to the extracellular level of the glucose analog of each red blood cell. In certain embodiments, the method further includes determining an average or median ratio of the intracellular level to the extracellular level of the glucose analog of the population of red blood cells or the subset thereof. In certain embodiments, the method further includes comparing the ratio or the average or median ratio to a reference ratio. In certain embodiments, the reference ratio is a value obtained from a plurality of subjects having at least one demographic metric in common with the patient. In certain embodiments, the reference ratio is a value obtained from a plurality of subjects having at least two demographic metrics in common with the patient. In certain embodiments, at least one of the demographic metrics is race. The present disclosure further includes methods of providing a personalized diagnosis and/or treatment to a patient using the ratio or the average or median ratio.

The present disclosure further provides kits and systems for performing the methods of the present disclosure.

### BRIEF DESCRIPTION OF THE FIGURES

The following figures are included to illustrate certain aspects of the present disclosure, and should not be viewed as exclusive embodiments. The subject matter disclosed is capable of considerable modifications, alterations, combinations and equivalents in form and function, without departing from the scope of this disclosure.

FIG. 1A provides the chemical structure of the glucose analog 2-NBDG (2-(N-(7-Nitrobenz-2-oxa-1,3-diazol-4-yl)Amino)-2-Deoxyglucose).

FIG. 1B provides a graph showing inhibition of the uptake of glucose analogs D-glucose-SiR and 6-NBDG and 2-NBDG (at 20 μM) in the presence of GLUT1 receptor inhibitors Phloretin, Cytochalasin B, WZB117, STF-31 and BAY-876. Average sample intensities were normalized to average intensity measured on RBCs exposed to 20 μM of one of three fluorescent glucose-analogs, respectively, and no inhibitor.

FIG. 2 provides a schematic showing an exemplary cell anchoring system for imaging of single cells. In FIG. 2, (1) represents a microscope glass coverslip, (2) represents a bovine serum albumin (BSA)-biotin conjugate, (3) represents a streptavidin-derivative (e.g., NeutrAvidin™), (4) represents an α-glycophorin-biotin conjugate and (5) represents the cell membrane of red blood cells (RBCs).

FIG. 3 provides spatial intensity maps of cells throughout the course of introduction and wash out of the glucose analog 2-NBDG.

FIG. 4 provides a region of interest (ROI) that encloses the RBCs and allow for intensity measurements at each image during a time-resolved image sequence.

FIG. 5 provides the kinetic details of the uptake of the glucose analog over a duration of about 5 minutes for each analyzed RBC. The dashed line represents the median intensity of the extracellular glucose analog (the background), and each other line represent the spectrum of uptake in which different RBCs experience during an experiment.

FIG. 6 provides the kinetic details of the same subset of RBCs at different glucose analog concentrations where dashed lines represent the median intensity of the extracellular glucose analog (the background), and each other line represents a cell within the experimental field of view. Colors of solid lines correspond to identical cells receiving different concentrations of glucose analog.

FIGS. 7A-7F provides three examples of cells that display different relative levels of uptake as compared to the background level of glucose tracer (5 mM) (FIGS. 7A-7C). Using a three-part heuristic model, we fit the linear portion of the uptake and the initial velocity (V₀) was determined, which was used in the Michaelis-Menten equation to determine the maximum velocity (Vₘₐₓ) of uptake for the GLUT1 receptor in RBCs when performed at various concentrations (FIGS. 7D-7F).

FIG. 8 provides histograms of the ratio of intracellular glucose to extracellular glucose from two different patients to determine the average ratio and the standard deviation. With each patient, repeated measurements of the same RBC subpopulation (e.g., population subset) were conducted using different glucose tracer concentrations during steady-state conditions (i.e., individual intracellular glucose saturation).

FIGS. 9A-9C show human erythrocyte homeostasis and glucose tracer-GLUT1 specificity testing. FIGS. 9A-9B show human erythrocyte membrane integrity tests using flow cytometry. FIG. 9A shows human erythrocytes exposed to Calcein AM (1 μM) for 60 minutes, washed in KCl solution lacking Calcein AM and measured on the flow cytometer at specified time intervals. Mean FITC-A intensities taken from forward and side scatter gated data were normalized to the maximum of all data gathered. Error bars are calculated % CV from three repeated measurements. FIG. 9B shows a subsample of human erythrocytes incubated with Calcein AM as described in FIG. 9A that were washed and then exposed to membrane perturbing stressors (either incubation of cells with KCL solution containing 5% DMSO or sonication for three minutes) and measured on the flow cytometer. Error bars are calculated % CV of normalized mean intensities over three repeated measurements. FIG. 9C shows washed human erythrocyte solutions that were incubated with 20 μM of one of three glucose analog tracers (D-glu-SiR, 6-NBDG, or 2-NBDG) containing a fixed concentration of unlabeled D-glucose for 60 minutes were then measured for average cell fluorescence intensity on a flow cytometer. The bars represent the normalized mean intensity for a given D-glucose concentration. As concentration of D-glucose increases, the intracellular 2-NBDG intensity continues to decrease indicating a strong correlation with the transportation of D-glucose using the GLUT1 transporter.

FIGS. 10A-10C provide an exemplary method of the present disclosure. FIG. 10A shows that the first step in the experimental protocol is the preparation of the microfluidic imaging plate with the cell anchoring system comprised of bovine serum albumin conjugated to a biotin molecule, NEUTRAVIDIN™, and then incubating red blood cells with α-glycophorin A+B antibodies. FIG. 10B shows that the next step is to load the microfluidic wells with the 2-NBDG solution and the “wash out” buffer (i.e., KCl buffer). Cells are then loaded prior to experiment by flowing cell suspension from well #8 into the observation window region of the microfluidic plate and allow cells to anchor to the anchor system. Then prime the 5 mM 2-NBDG solution by flowing for 3-5 (depending on experiment) minutes, then washing out the tracer with 125 mM KCl buffer for 3-3.5 (depending on experiment) minutes. FIG. 10C presents a flow diagram depiction of the routine performed for a glucose uptake measurement for a single field of view containing adherent cells. This routine is either repeated for several fields of view (indicated by the “Start” label), or terminated, which then proceeds to the image analysis step (indicated by “End” label).

FIGS. 11A-11C provide exemplary data acquired from flow-based experiment showing the intra-patient variability in erythrocyte glucose uptake. FIG. 11A provides exemplary spatial intensity maps acquired at different times throughout a typical 2-NBDG uptake experiment. Background intensity increases relative to cellular cross sections upon tracer flow in, which is then maintained for 100 seconds. After 100 seconds, 2-NBDG is exchanged with KCl solution lacking 2-NBDG, which washes out tracer from the field of view. FIG. 11B provides a sub-region of spatial intensity maps taken from FIG. 11A showing magnified cells which have had the background removed, and the intensity amplified to show the intracellular region intensity following the same pattern as presented in FIG. 11A. FIG. 11C provides data from FIG. 11A (i.e., a subset of 43 cellular regions of interest) shown as median intracellular glucose percentage (or intracellular glucose (%) for simplicity) versus time in seconds (shown as solid-colored lines). Cells from a typical patient have intracellular glucose (%) ranging from 1.2% to 19.7%. FIG. 11C also contains an inset (top right) which shows the background region of interest (black dashed line) relative to the 43 example cells. The mean intracellular percentage for this patient blood sample was 4.9%.

FIGS. 12A-12C show intracellular glucose percentage stability and inter-sample stability. FIG. 12A provides five randomly chosen cells from a given field of view (FOV) that are plotted as intracellular glucose percentage (%) for each measurement taken throughout a time series experiment. Labeled 1-5, the sub-panels represent sequential repeats of said experiment and the identical cells being analyzed repeatedly. FIG. 12B provides histograms of intracellular glucose percentage for all cells taken from a single field of view (FOV). The legend shows the color which corresponds to the repeated experiment described in FIG. 12A. Overlapping histograms are portrayed by the dark blue regions. FIG. 12C provides the mean of the intracellular glucose percentage histogram was calculated for three separate patients using the method described herein. The same samples from each patient were then repeated three separate times (i.e., 0 hours, 24 hours, and 48 hours apart). Mean intracellular glucose percentage was calculated from a patient histogram on each day and tabulated as a single value.

FIGS. 13A-13B provide long duration saturation stability testing of intracellular 2-NBDG. FIG. 13A shows human erythrocytes prepared as described herein with only modification to imaging frequency. The initial flow in of 2-NBDG is imaged as described herein, but once the saturation plateau is achieved, sequential imaging occurs every six minutes for a total of 21 images (or ˜120 minutes total), followed by identical wash away imaging protocol. The plot of median intracellular fluorescence intensity versus time shows several typical cells at various levels (multiple color solid lines) of relative intracellular glucose fraction maintaining their saturation plateau across the image sequencing occurring over one hour with minimal variation. This control shows the plateau is not an artifact, but a property of the cell reacting with the 2-NBDG tracer. FIG. 13B provides the same plot shown in FIG. 13A zoomed out to compare cells intensities with that of the background (dashed black line).

FIGS. 14A-14B provide example distributions of intracellular glucose fraction. FIG. 14A provides the median intensity value for each cellular ROI, taken from the optimized z-plane image during tracer saturation from each field of view, were gathered and normalized to the background intensity for said field-of-view. This computed value becomes the intracellular glucose fraction. All cells from each field of view for a given experiment are then collected, binned into 0.01 intervals, and counted to form the frequency histogram of intracellular glucose fraction. The mean of the distribution is then computed, such that FIG. 14A represents a typical patient with a lower intracellular glucose fraction ( [symbol] a [symbol] =4.6%±1.6%, 2343 cells, mean±1 SD) and FIG. 14B represents a typical patient with a higher intracellular glucose fraction ( [symbol] b [symbol] =6.7%±2.3%, 1802 cells, mean±1 SD). These histograms were filtered using cell heights ≥2.1 μm.

FIGS. 15A-15B provides a cartoon depiction and graphical depiction of median fluorescence intensity of a typical erythrocyte taken at several objective heights. FIG. 15A provides a cartoon depiction of a cell anchored to a microscope cover slide surrounded by 2-NBDG tracer solution. FIG. 15B provides a graph showing a representative plot of median tracer fluorescence intensity versus objective height for an average height erythrocyte (z-profiles). Open black circles represent the data, solid gray line is the running average of the data across five data point. Open red circles are the objective heights identified as the lower cell membrane (objective height 1.9 μm, leftmost red circle), and the upper membrane (objective height 4.5 μm, rightmost red circle), for which the difference provides an estimated cell height of 2.6 μm. The blue circle with an “x” indicates the height selected by the height selection routine described herein.

FIGS. 16A-16D provide an analysis of mean intracellular glucose fractions pooled by demographic identifier that reveals statistically significant variation in glucose-tracer uptake. FIG. 16A shows that for cells of height greater than 2.1 μm, each patient sample mean intracellular glucose fraction was pooled by their race (i.e., Black or Hispanic/Caucasian) and plotted into a Box-Whisker plot (black dots are means intracellular fraction data for each patient in random order). FIG. 16B shows that for cells inclusive of the z-height correction factor, each patient sample mean intracellular glucose fraction was pooled by their race (i.e., Black or Hispanic/Caucasian) and plotted into a Box-Whisker plot (black dots are means intracellular fraction data for each patient in random order). FIGS. 16C-16D shows paired t-test results for all patients shown in FIG. 16A and FIG. 16B, respectively. The mean of the pooled data shows a difference of 0.006 between categories (or 22.81% difference) for data in FIG. 16A, and 0.016 between categories (or 26.67% difference) for data in FIG. 16B. These data sets were tested for significance using the two-sample, equal variance, T-test (see Table 2 for results) leading to rejection of the null hypotheses under both analysis criteria (z-height rejection, or z-height corrected).

FIG. 17 provides normalized intracellular 2-NDBG relative to overall mean fraction and by racial groups. Circle points on the box-whisker plot represent the group means.

FIG. 18 provides an analysis of mean intracellular glucose percentages pooled by demographic identifier, which reveals high variability in glucose-tracer uptake. For cells (height corrected), each donor sample mean intracellular glucose percentage (black diamonds) was pooled by their race (i.e., Black or Hispanic/Caucasian), normalized to the mean intracellular glucose percentage of the entire study, or all donor mean (red dashed line), and plotted into a Box-Whisker plot (boxes encompass 25% to 75% of all data, whiskers represent coefficient of 1 for outliers, line inside box represents the population median, and white circle inside box is the population mean). As shown, the mean of the Black subgroup is 1.18 (+16.51% difference from the all donor mean), while the mean of the Hispanic/Caucasian subgroup is 0.86 (−15.05% difference from the all donor mean). The total number of donors in the study (N) is 45.

### DETAILED DESCRIPTION

The present disclosure relates to methods for determining the kinetics of glucose uptake in individual red blood cells (RBCs) obtained from subjects. In certain embodiments, the methods of the present disclosure are in vitro methods. The present disclosure is based, in part, on the findings that the kinetics of glucose uptake can vary among individual patients. For example, and as described herein, certain characteristics of a patient, including race, influence the rate of glucose internalization and/or the levels of intracellular glucose.

The present disclosure provides methods for determining the kinetics of glucose uptake in individual red blood cells (RBCs) obtained from subjects, e.g., which can allow for more individualized diagnoses and treatment. For example, but not by way of limitation, methods of the present disclosure can be used to determine intracellular glucose levels, rate of glucose uptake and the ratio of intracellular glucose to extracellular glucose of individual RBCs. Such information can be used, for example, patient personalized diagnoses, treatments and/or monitoring protocols of diabetes. In certain embodiments, information obtained from the methods of the present disclosure can be used to personalize a patient's HbA1c score. The present disclosure further provides kits and systems for performing the methods described herein.

For clarity, but not by way of limitation, the detailed description of the presently disclosed subject matter is divided into the following subsections:
I. Definitions;
II. Methods;
III. Clinical Uses;
IV. Kits;
V. Systems; and
VI. Exemplary Embodiments.

### I. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately,” as used herein, can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” can mean an acceptable error range for the particular value, such as +10% of the value modified by the term “about.”

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{d}). Affinity can be measured by common methods known in the art.

The term “antigen-binding molecule” herein is used in the broadest sense and encompasses various peptides, proteins, antibody structures, including but not limited to monoclonal antibodies, polyclonal antibodies, multispecific antibodies (e.g., bispecific antibodies) and antibody fragments so long as they exhibit the desired antigen-binding activity.

The term “antibody” herein is used in the broadest sense and encompasses various antibody structures, including but not limited to monoclonal antibodies, polyclonal antibodies, multispecific antibodies (e.g., bispecific antibodies) and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g., scFv) and multispecific antibodies formed from antibody fragments.

The term “coupled” can refer to the connecting or uniting of two or more components by an interaction, bond, link, force or tie in order to keep two or more components together. In certain embodiments, the term “coupled” encompasses either direct or indirect binding where, for example, a first component is directly bound to a second component, or one or more intermediate molecules are disposed between the first component and the second component. Exemplary bonds comprise covalent bonds, ionic bonds, van der Waals interactions and other bonds identifiable by a skilled person. The term “detecting,” is used herein, to include both qualitative and quantitative measurements of a target molecule, e.g., a glucose analog, or processed forms thereof. In certain embodiments, detecting includes identifying the mere presence of the target molecule as well as determining whether the target molecule is present at detectable levels.

The terms “HbA1c level,” “HbA1c value” and “HbA1c” are used interchangeably herein.

As used herein, the term “imaging” refers to microscopy. In certain embodiments, microscopy includes immunofluorescence microscopy.

An “individual,” “subject” or “patient” herein is a vertebrate, such as a human or non-human animal, for example, a mammal. Mammals include, but are not limited to, humans, non-human primates, farm animals, sport animals, rodents and pets.

Non-limiting examples of non-human animal subjects include rodents such as mice, rats, hamsters, and guinea pigs; rabbits; dogs; cats; sheep; pigs; goats; cattle; horses; and non-human primates such as apes and monkeys. In certain embodiments, the individual, subject or patient is a human.

As used herein, the term “inhibit” means to reduce, inhibit, block and/or eliminate.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

The term “isolated,” as used herein, denotes a degree of separation from original source or surroundings.

The terms “label” or “detectable label,” as used herein, refers to any chemical group or moiety that can be linked to a substance that is to be detected or quantitated. A label is a detectable label that is suitable for the sensitive detection or quantification of a substance. Non-limiting examples of detectable labels include, but are not limited to, luminescent labels, e.g., fluorescent, phosphorescent, chemiluminescent, bioluminescent and electrochemiluminescent labels, radioactive labels, enzymes, particles, magnetic substances, electroactive species and the like. Alternatively, a detectable label can signal its presence by participating in specific binding reactions. Non-limiting examples of such labels include haptens, antibodies, biotin, streptavidin, his-tag, nitrilotriacetic acid, glutathione S-transferase, glutathione and the like.

The terms “plurality” or “population” refers to a number larger than one. In certain embodiments, the term “plurality of cells” or “population of cells” refers to a number of cells larger than one. For example, but not by way of limitation, a plurality of cells includes at least two cells. In certain embodiments, a plurality of cells includes about 2 or more cells, about 10 or more cells, about 50 or more cells, about 100 or more cells, about 1,000 or more cells or about 10,000 or more cells.

The terms “polypeptide” and “protein,” as used interchangeably herein, refer to polymers of amino acids of any length. The polymer may be linear or branched, it may comprise modified amino acids, and it may be interrupted by non-amino acids. The terms also encompass an amino acid polymer that has been modified naturally or by intervention;

for example, disulfide bond formation, glycosylation, lipidation, acetylation, phosphorylation or any other manipulation or modification, such as conjugation with a labeling component. Also included within the definition are, for example, polypeptides containing one or more analogs of an amino acid (including, for example, unnatural amino acids, etc.), as well as other modifications known in the art. The terms “polypeptide” and “protein” as used herein specifically encompass antibodies.

The term “specifically binds,” as used herein, refers to the preferential binding to a target molecule, e.g., a protein, relative to other molecules, e.g., proteins, in a sample or on a surface of an RBC.

As used herein, the term “subset” refers to a small portion of a larger quantity of material, e.g., a subset of a larger quantity of cells.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state and remission or improved prognosis. In certain embodiments, methods of the present disclosure can be used to obtain information that can inform the treatment of pre-diabetic or diabetic patients.

As described herein, any concentration range, percentage range, ratio range or integer range is to be understood to include the value of any integer within the recited range and, when appropriate, fractions thereof (such as one tenth and one hundredth of an integer), unless otherwise indicated.

Other aspects of the presently disclosed subject matter are described in the following disclosure and are within the ambit of the presently disclosed subject matter.

### II. Methods

The present disclosure relates to methods for determining the kinetics of glucose uptake in individual RBCs obtained from subjects. In certain embodiments, the methods of the present disclosure are in vitro methods. For example, but not by way of limitation, methods of the present disclosure can be used to determine intracellular glucose levels, rate of glucose uptake and the ratio of intracellular glucose to extracellular glucose (also referred to herein as intracellular glucose percentage) of individual RBCs and for a population of RBCs. In certain embodiments, the present disclosure provides a method for determining intracellular glucose levels, e.g., of individual RBCs and for a population of RBCs (e.g., of a patient). In certain embodiments, the present disclosure provides a method for determining the ratio of intracellular glucose to extracellular e.g., of individual RBCs and for a population of RBCs (e.g., of a patient). In certain embodiments, the present disclosure provides a method for determining rates of glucose uptake, e.g., of individual RBCs and for a population of RBCs (e.g., of a patient). In certain embodiments, the RBCs are obtained from a patient (e.g., a single patient). In certain embodiments, the RBCs are obtained from a sample of a patient (e.g., a sample of a single patient).

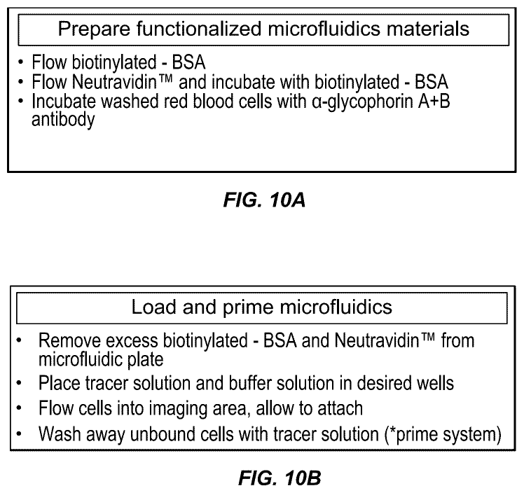

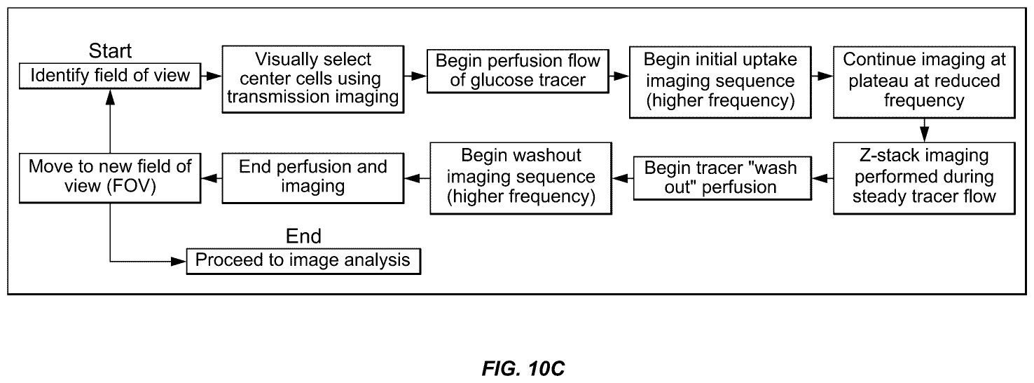

An exemplary method of the present disclosure is provided in FIG. 10. The presently disclosed methods provide a versatile, yet simple, way of measuring uptake of small molecules by cellular transporters such as glucose transporter 1 (GLUT1).

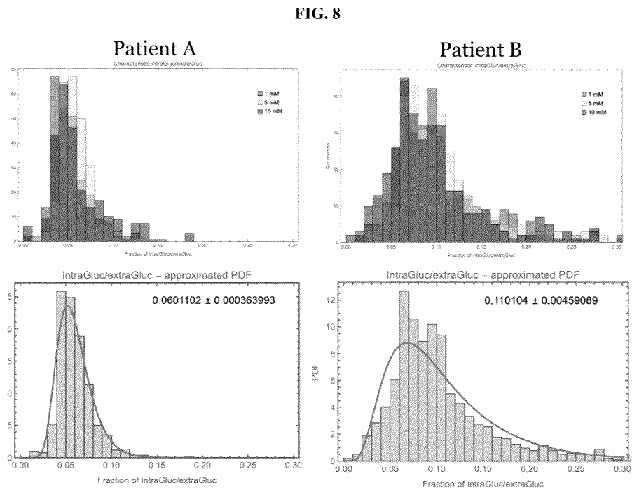

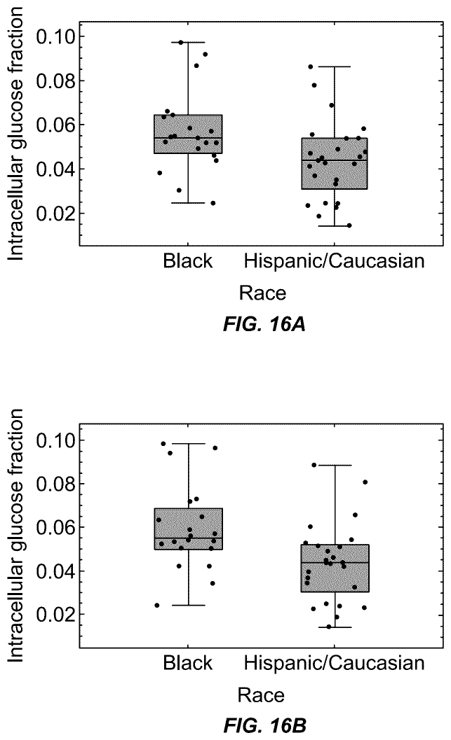

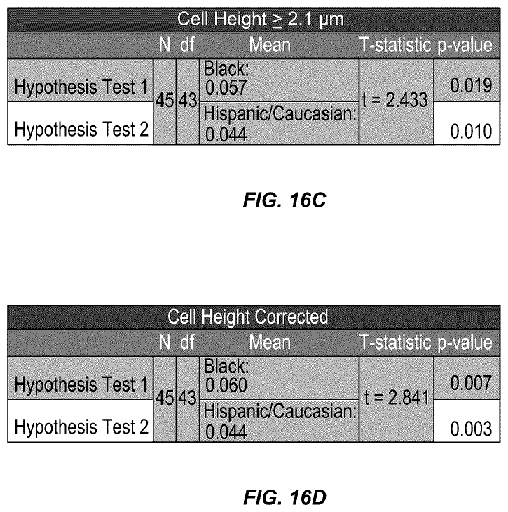

As shown in FIG. 8, significant differences in RBC glucose kinetics and glucose intracellular levels are observed between different patients, and this information can be used to personalize diagnoses, treatments and/or monitoring protocols, e.g., of pre-diabetic or diabetic patients. For example, as shown in FIGS. 16-18, significant differences in RBC glucose kinetics and glucose intracellular levels are observed between patients of different races. In certain embodiments, the kinetics of glucose uptake can be used to personalize a patient's HbA1c score, to monitor progress and/or effectiveness of a patient's personalized diabetes management, alter a patient's personalized diabetes management, identify an abnormal or diseased physiological condition and/or identify patients taking supplements and/or therapeutics that effect red blood cell uptake of glucose (e.g., as disclosed in International Patent Publication WO 2021/108419 (e.g., at paragraphs [0135]-[0166]), the contents of which is incorporated herein by reference in its entirety for all purposes).

In certain embodiments, the method for determining the kinetics of glucose uptake in individual RBCs includes providing RBCs, e.g., a population of RBCs. In certain embodiments, the RBCs for use in the present disclosure can be obtained from any source. In certain embodiments, the RBCs for use in the present disclosure are obtained from a patient. In certain embodiments, the RBCs for use in the present disclosure are obtained from a single patient. In certain embodiments, the RBCs for use in the present disclosure are obtained from a sample of a patient. In certain embodiments, the RBCs for use in the present disclosure are obtained from a sample of a single patient. In certain embodiments, the RBCs can be obtained from a fresh patient blood sample, e.g., isolated from a fresh patient blood sample. In certain embodiments, the RBCs can be obtained from preserved samples, e.g., from frozen samples, e.g., from frozen patient samples. In certain embodiments, the RBCs can be isolated from a sample of a patient, e.g., a fresh or frozen sample of a patient, by any method known in the art, e.g., by a density gradient centrifugation. In certain embodiments, the patient is a human or a non-human primate such as an ape and a monkey. In certain embodiments, the individual or patient is a human. In certain embodiments, the RBCs can be isolated from a blood sample, e.g., a whole blood sample, by centrifugation. Alternatively, the RBCs can be obtained from in vitro cell cultures. For example, but not by way of limitation, RBCs for use in the present disclosure can be differentiated from stem cells, e.g., hematopoietic stem cells, or induced pluripotent stem cells (iPSCs). In certain embodiments, the stem cells can be obtained from a patient and/or the iPSCs can be obtained by reprogramming cells (e.g., somatic cells) from a patient.

In certain embodiments, the RBCs for use in the present disclosure can be treated with an agent, e.g., a therapeutic agent. In certain embodiments, the RBCs can be genetically modified, e.g., genetically modified with a gene editing system. Non-limiting examples of gene editing systems include homing endonucleases or meganucleases, zinc finger nucleases (ZFN), transcription activator-like effector nucleases (TALENs) and CRISPR gene editing system. For example, but not by way of limitation, the RBCs can be genetically modified to eliminate or reduce expression of a gene, e.g., eliminate or reduce expression of a gene that is relevant to a disease (e.g., causes a disease). In certain embodiments, the RBCs can be genetically modified to increase or enhance the expression of a gene, e.g., to increase or enhance expression of a gene that is relevant to a disease (e.g., causes a disease).

In certain embodiments, a population of RBCs can include at least about 5 RBCs. For example, but not by way of limitation, the population of RBCs includes about 10 or more RBCs, about 20 or more RBCs, about 30 or more RBCs, about 40 or more RBCs, about 50 or more RBCs, about 60 or more RBCs, about 70 or more RBCs, about 80 or more RBCs, about 90 or more RBCs, about 100 or more RBCs, about 150 or more RBCs, about 200 or more RBCs, about 300 or more RBCs, about 400 or more RBCs, about 500 or more RBCs, about 1,000 or more RBCs, about 2,000 or more RBCs, about 3,000 or more RBCs, about 4,000 or more RBCs, about 5,000 or more RBCs, about 6,000 or more RBCs, about 7,000 or more RBCs, about 8,000 or more RBCs, about 9,000 or more RBCs or about 10,000 or more RBCs. In certain embodiments, a population of RBCs includes about 5 about 10,000 red blood cells.

In certain embodiments, a population of RBCs includes about 100 to about 5,000 red blood cells.

In certain embodiments, a population of RBCs includes at least about 100 RBCs.

In certain embodiments, a population of RBCs includes at least about 350 RBCs.

In certain embodiments, a population of RBCs includes at least about 1,000 RBCs.

In certain embodiments, a population of RBCs includes at least about 10,000 RBCs.

In certain embodiments, the concentration of RBCs used in the presently disclosed methods can depend on the size of the wells, chamber, culture dishes and/or plates used. For example, but not by way of limitation, RBCs can be used in the presently disclosed method at a concentration from about 1×10² to about 1×10⁵ cells/ml. In certain embodiments, the number of RBCs used can be from about 1×10² to about 1×10⁴ cells/ml, from about 1×10² to about 1×10³ cells/ml, from about 1×10³ to about 1×10⁵ cells/ml, from about 1×10⁴ to about 1×10⁵ cells/ml or from about 1×10³ to about 1×10⁴ cells/ml. In certain embodiments, about 1×10³ cells/ml RBCs are used. In certain embodiments, the number of RBCs used can be from about 1×10² cells/ml to about 1×10⁴ cells/ml. In certain embodiments, RBCs can be used at a concentration from about 1×10² cells to about 1×10³ cells/ml.

In certain embodiments, RBCs analyzed in the presently disclosed methods are of a certain height. For example, but not by way of limitation, RBCs analyzed in the presently disclosed methods are of a certain cell height. In certain embodiments, the height of a cell is the length (e.g., diameter) of the cell that is perpendicular to the substrate (e.g., along the z-axis as described in Example 2). In certain embodiments, the RBCs have a cell height greater than about 2.0 μm, e.g., greater than about 2.1 μm, greater than about 2.2 μm, greater than about 2.3 μm, greater than about 2.4 μm, greater than about 2.5 μm, greater than about 2.6 μm, greater than about 2.7 μm, greater than about 2.8 μm, greater than about 2.9 μm or greater than about 3.0 μm. In certain embodiments, the RBCs have a cell height greater than about 2.0 μm. In certain embodiments, the RBCs have a cell height greater than about 2.1 μm. In certain embodiments, the RBCs have a minimum cell height of about 2.0 μm or greater, e.g., about 2.1 μm or greater, about 2.2 μm or greater, about 2.3 μm or greater, about 2.4 μm or greater, about 2.5 μm or greater, about 2.6 μm or greater, about 2.7 μm or greater, about 2.8 μm or greater, about 2.9 μm or greater or about 3.0 μm or greater.

In certain embodiments, RBCs analyzed in the presently disclosed methods have a minimum cell height of about 2.1 μm.

In certain embodiments, RBCs analyzed in the presently disclosed methods have a cell height of at least about 2.1 μm or greater.

In certain embodiments, the method can further include anchoring the RBCs to a substrate. In certain embodiments, the substrate can be any surface useful in imaging and/or retaining the RBCs. In certain embodiments, the substrate can be a glass or plastic substrate. Non-limiting examples of substrates include a well (e.g., a well of a cell culture dish or plate), a chamber (e.g., a chamber of a microfluidic device) and a coverslip (e.g., a glass coverslip). In certain embodiments, the substrate is present within or is a part of a microfluidic device. In certain embodiments, the substrate can be a cell culture chamber of a microfluidic device or a coverslip within a cell culture chamber of a microfluidic device.

In certain embodiments, RBCs are anchored to the substrate to allow imaging of the same RBCs for a length of time. In certain embodiments, RBCs are anchored to the substrate to allow imaging of the same RBCs (e.g., at the same spatial location on the substrate) for at least about 30 seconds, at least about 1 minute, at least about 2 minutes, at least about 3 minutes, at least about 4 minutes, at least about 5 minutes, at least about 6 minutes, at least about 7 minutes, at least about 8 minutes, at least about 9 minutes, at least about 10 minutes, at least about 11 minutes, at least about 12 minutes, at least about 13 minutes, at least about 14 minutes, at least about 15 minutes, at least about 16 minutes, at least about 17 minutes, at least about 18 minutes, at least about 19 minutes or at least about 20 minutes. In certain embodiments, RBCs are anchored to the substrate to allow imaging of the same RBCs (e.g., at the same spatial location on the substrate) for a duration of about 1 to about 30 minutes, e.g., about 1 to about 25 minutes, about 1 to about 20 minutes, about 1 to about 15 minutes, about 1 to about 10 minutes, about 1 to about 9 minutes, about 1 to about 8 minutes, about 1 to about 7 minutes, about 1 to about 6 minutes, about 1 to about 5 minutes, about 1 to about 4 minutes or about 1 to about 3 minutes. In certain embodiments, RBCs are anchored to the substrate to allow imaging of the same RBCs (e.g., at the same spatial location on the substrate) for a duration of about 1 to about 6 minutes. In certain embodiments, RBCs are anchored to the substrate to allow imaging of the same RBCs (e.g., at the same spatial location on the substrate) for a duration of about 1 to about 10 minutes. In certain embodiments, RBCs are anchored to the substrate to allow imaging of the same RBCs (e.g., at the same spatial location on the substrate) for a duration of about 5 to about 10 minutes. In certain embodiments, RBCs are anchored to the substrate to allow imaging of the same RBCs (e.g., at the same spatial location on the substrate) for a duration of about 1 to about 20 minutes. In certain embodiments, RBCs are anchored to the substrate to allow imaging of the same RBCs (e.g., at the same spatial location on the substrate) for a duration of about 5 to about 20 minutes.

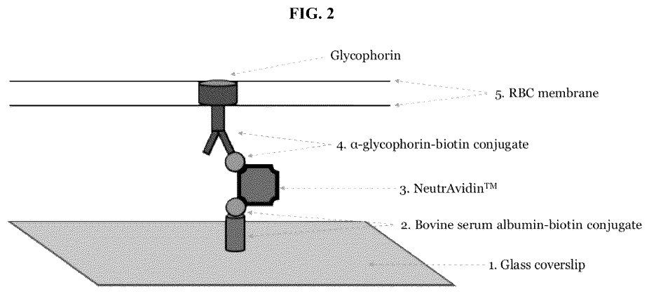

In certain embodiments, the RBCs are anchored to the substrate using an antigen-binding based anchoring approach (e.g., an antibody-based anchoring approach), an avidin-biotin anchoring approach or a combination thereof. A non-limiting exemplary embodiment of an anchoring system is shown in FIG. 2. In certain embodiments, the substrate is coated with a serum albumin-biotin conjugate, e.g., a bovine serum albumin-biotin conjugate. In certain embodiments, the serum albumin-biotin conjugate is coupled to an avidin or an analog thereof. In certain embodiments, the avidin analog is a deglycosylated form of avidin. In certain embodiments, the avidin analog is a deglycosylated form of avidin that has a mass of about 60,000 Da. In certain embodiments, the avidin analog is a deglycosylated form of avidin that lacks the four mannose and three N-acetyl glucosamine residues in each unit. In certain embodiments, the avidin analog is NEUTRAVIDIN™. In certain embodiments, the substrate is coated with a cell anchoring system (e.g., that includes, at least, a serum albumin-biotin conjugate and an avidin or analog thereof). In certain embodiments, the substrate is coated with a cell anchoring system that includes a serum albumin-biotin conjugate coupled to an avidin or analog thereof.

In certain embodiments, the substrate is initially coated with the serum albumin-biotin conjugate of the cell anchoring system. In certain embodiments, the substrate is coated with the serum albumin-biotin conjugate by contacting the substrate with the serum albumin-biotin conjugate at a concentration from about 0.01 mg/ml to about 10 mg/ml, e.g., about 0.1 mg/ml. In certain embodiments, the substrate can be contacted with the serum albumin-biotin conjugate for about 5 minutes to about 1 hour, e.g., about 15 minutes, at a temperature from about 10° C. to about 37° C., e.g., about 25° C. In certain embodiments, the substrate coated with the serum albumin-biotin conjugate can then be contacted with the avidin (or avidin analog) (e.g., of the cell anchoring system), e.g., at a concentration from about 0.01 mg/ml to about 10 mg/ml, e.g., about 0.1 mg/ml. In certain embodiments, the substrate coated with the serum albumin-biotin conjugate can then be contacted with the avidin (or avidin analog) for about 5 minutes to about 1 hour, e.g., about 15 minutes, at a temperature from about 10° C. to about 37° C., e.g., about 25° C., to form the anchoring system.

In certain embodiments, the RBCs are contacted with an antigen-binding molecule coupled to biotin, which is also referred to herein as an “antigen-binding molecule-biotin conjugate.” In certain embodiments, the antigen-binding molecule can be a peptide that specifically binds to the antigen, a protein or fusion protein that specifically binds to the antigen or an antibody or an antibody fragment that specifically binds to the antigen. In certain embodiments, the antigen-binding molecule is an antibody that is specific for the antigen (e.g., an antibody-biotin conjugate). In certain embodiments, the affinity between the antibody and the antigen is characterized by a dissociation constant (K_{d}) of ≤1 M, ≤100 mM, ≤10 mM, ≤1 mM, ≤100 μM, ≤10 μM, ≤1 μM, ≤100 nM, ≤10 nM, ≤1 nM, ≤0.1 nM, ≤0.01 nM or ≤0.001 nM. In certain embodiments, the antibody specific for the antigen can have a K_{d} of about 10⁻³ or less, or 10⁻⁸ M or less, e.g., from 10⁻⁸ M to 10⁻¹³ M, e.g., from 10⁻⁹ M to 10⁻¹³ M. In certain embodiments, the antibody can be an antibody fragment, e.g., a Fab, Fab′, Fab′-SH, F(ab′)₂, Fv, scFv, diabody or a single-domain antibody.

In certain embodiments, the antigen-binding molecule binds to an antigen on the surface of an RBC. In certain embodiments, the antigen-binding molecule binds to an antigen on the surface of an RBC without affecting the activity of the GLUT1 receptor. GLUT1 is responsible for facilitated transport of glucose across the plasma membrane in several mammalian cells and is part of the solute carrier family 2. In human RBCs, this transporter is responsible for maintaining the cellular influx and efflux of glucose. In certain embodiments, the antigen-binding molecule binds to a protein on the surface of an RBC without affecting the activity of the GLUT1 receptor. In certain embodiments, the protein can be a transmembrane protein or a protein that is anchored to the membrane of the RBC. In certain embodiments, the RBC surface proteins that can be used to anchor RBCs to a substrate include membrane transporters, membrane channels, receptors, adhesion molecules, enzymes and structural proteins. Non-limiting examples of surface proteins that can be used to anchor RBCs to a substrate include Urea transporter (UT)-B, Aquaporin 1, Aquaporin 3, Rh-associated glycoprotein (CD241), CD59, CD47, Lutheran glycoprotein (CD329), Intercellular adhesion molecule-4 (ICAM4), ADP-ribosyltransferase 4 (CD297), Kell glycoprotein (CD238), Semaphorin 7A (CD108), Acetylcholinesterase, Erythroblast membrane-associated protein (ERMAP), Glycophorin A (CD235A), Glycophorin B (CD235B), Glycophorin C (CD236C), Glycophorin D (CD236D) and Xg glycoprotein. Additional non-limiting examples are disclosed in Daniels, Vox Sanguinis 93:331-340 (2007) (e.g., Table 1), the contents of which is incorporated by reference herein in its entirety. In certain embodiments, the antigen-binding molecule binds to a glycophorin, e.g., Glycophorin A, Glycophorin B, Glycophorin C and/or Glycophorin D, on the surface of RBCs. In certain embodiments, the antigen-binding molecule (e.g., antibody) binds to Glycophorin A on the surface of RBCs. In certain embodiments, the antigen-binding molecule (e.g., antibody) binds to Glycophorin B on the surface of RBCs. In certain embodiments, the antigen-binding molecule (e.g., antibody) binds to Glycophorin A and Glycophorin B on the surface of RBCs. In certain embodiments, the antigen-binding molecule (e.g., antibody) binds to an N-terminal region of Glycophorin A and/or Glycophorin B on the surface of RBCs. In certain embodiments, the antigen-binding molecule (e.g., antibody) binds to an N-terminal region of Glycophorin A and Glycophorin B on the surface of RBCs. A non-limiting example of a biotin-labeled antigen-binding molecule (e.g., antibody) that binds to Glycophorin A and Glycophorin B is Cat No. ab 15009 from ABCAM plc. In certain embodiments, the antigen-binding molecule (e.g., antibody) does not bind to the GLUT1 receptor. In certain embodiments, the antigen-binding molecule (e.g., antibody) does not affect the activity of the GLUT1 receptor. In certain embodiments, the antigen-binding molecule (e.g., antibody) does not bind to the GLUT1 receptor and does not affect the activity of the GLUT1 receptor.

In certain embodiments, the RBCs are contacted with the antigen-binding molecule-biotin conjugate (e.g., the antibody-biotin conjugate) at a concentration of about 0.001 mg/ml to about 1 mg/ml, e.g., about 0.001 mg/ml to about 0.9 mg/ml, about 0.001 mg/ml to about 0.8 mg/ml, about 0.001 mg/ml to about 0.7 mg/ml, about 0.001 mg/ml to about 0.6 mg/ml, about 0.001 mg/ml to about 0.5 mg/ml, about 0.001 mg/ml to about 0.4 mg/ml, about 0.001 mg/ml to about 0.3 mg/ml, about 0.001 mg/ml to about 0.2 mg/ml, about 0.001 mg/ml to about 0.1 mg/ml, about 0.001 mg/ml to about 0.05 mg/ml, about 0.001 mg/ml to about 0.01 mg/ml, about 0.001 mg/ml to about 0.005 mg/ml, about 0.005 mg/ml to about 1 mg/ml, about 0.01 mg/ml to about 1 mg/ml, about 0.05 mg/ml to about 1 mg/ml, about 0.1 mg/ml to about 1 mg/ml or about 0.05 mg/ml to about 0.5 mg/ml. In certain embodiments, the RBCs are contacted with the antigen-binding molecule-biotin conjugate (e.g., the antibody-biotin conjugate) at a concentration of about 0.001 mg/ml to about 0.01 mg/ml, e.g., about 0.0025 mg/ml. In certain embodiments, the RBCs are contacted with the antigen-binding molecule-biotin conjugate (e.g., the antibody-biotin conjugate) at a concentration of about 0.01 mg/ml to about 1 mg/ml, e.g., about 0.01 mg/ml to about 0.9 mg/ml, about 0.01 mg/ml to about 0.8 mg/ml, about 0.01 mg/ml to about 0.7 mg/ml, about 0.01 mg/ml to about 0.6 mg/ml, about 0.01 mg/ml to about 0.5 mg/ml, about 0.01 mg/ml to about 0.4 mg/ml, about 0.01 mg/ml to about 0.3 mg/ml, about 0.01 mg/ml to about 0.2 mg/ml, about 0.01 mg/ml to about 0.1 mg/ml, about 0.02 mg/ml to about 1 mg/ml, about 0.03 mg/ml to about 1 mg/ml, about 0.04 mg/ml to about 1 mg/ml, about 0.05 mg/ml to about 1 mg/ml, about 0.06 mg/ml to about 1 mg/ml, about 0.07 mg/ml to about 1 mg/ml, about 0.08 mg/ml to about 1 mg/ml, about 0.09 mg/ml to about 1 mg/ml, about 0.1 mg/ml to about 1 mg/ml or about 0.05 mg/ml to about 0.5 mg/ml.

In certain embodiments, the RBCs are contacted with the antigen-binding molecule-biotin conjugate at a concentration of about 0.05 mg/ml to about 0.5 mg/ml, e.g., about 0.1 mg/ml.

In certain embodiments, the RBCs are contacted with the antigen-binding molecule-biotin conjugate (e.g., the antibody-biotin conjugate) from about 30 minutes to about 5 hours, e.g., about 30 minutes to about 3 hours, from about 30 minutes to about 2 hours or from about 30 minutes to about 1.5 hours, e.g., about 1 hour.

In certain embodiments, the RBCs are contacted with the antigen-binding molecule-biotin conjugate (e.g., the antibody-biotin conjugate) at a temperature of about 25° C. to about 45° C., e.g., about 37° C.

In certain embodiments, anchoring the RBCs to the substrate can include contacting the coated substrate with the RBCs that were incubated with the antigen-binding molecule-biotin conjugate (e.g., the antibody-biotin conjugate). In certain embodiments, the biotin of the antigen-binding molecule-biotin conjugate (e.g., the antibody-biotin conjugate) interacts with the avidin (or avidin analog) coating the substrate to anchor the RBCs to the substrate. In certain embodiments, contacting the coated substrate with the RBCs that were incubated with the antigen-binding molecule-biotin conjugate (e.g., the antibody-biotin conjugate) can include flowing the RBCs over the coated substrate, e.g., using a microfluidic device. In certain embodiments, the RBCs that are bound by the antigen-binding molecule-biotin conjugate (e.g., the antibody-biotin conjugate) are contacted with the coated substrate by perfusion, e.g., the RBCs that are bound by the antigen-binding molecule-biotin conjugate (e.g., the antibody-biotin conjugate) are flowed across the coated substrate at a perfusion rate that allows the anchoring of the RBCs to the substrate. In certain embodiments, the perfusion rate is from about 1 μL/hr to about 500 μL/hr, e.g., about 1 μL/hr to about 400 μL/hr, about 1 μL/hr to about 300 μL/hr, about 1 μL/hr to about 200 μL/hr, about 1 μL/hr to about 100 μL/hr, about 1 μL/hr to about 50 μL/hr, about 1 μL/hr to about 40 μL/hr, about 1 μL/hr to about 30 μL/hr, about 5 μl/hr to about 100 μL/hr, about 10 μL/hr to about 100 μL/hr, about 10 μL/hr to about 50 μL/hr or about 10 μL/hr to about 30 μL/hr.

In certain embodiments, the RBCs that are bound by the antigen-binding molecule-biotin conjugate (e.g., the antibody-biotin conjugate) are flowed across the coated substrate at a perfusion rate from about 1 μL/hr to about 50 μL/hr.

In certain embodiments, the RBCs that are bound by the antigen-binding molecule-biotin conjugate (e.g., the antibody-biotin conjugate) are flowed across the coated substrate at a perfusion rate from about 10 μL/hr to about 30 μL/hr.

In certain embodiments, the RBCs that are bound by the antigen-binding molecule-biotin conjugate (e.g., the antibody-biotin conjugate) are flowed across the coated substrate at a perfusion rate from about 20 μL/hr.

In certain embodiments, the RBCs that are bound by the antigen-binding molecule-biotin conjugate (e.g., the antibody-biotin conjugate) are contacted with the coated substrate from about 1 minute to about 30 minutes, e.g., about 3 minutes.

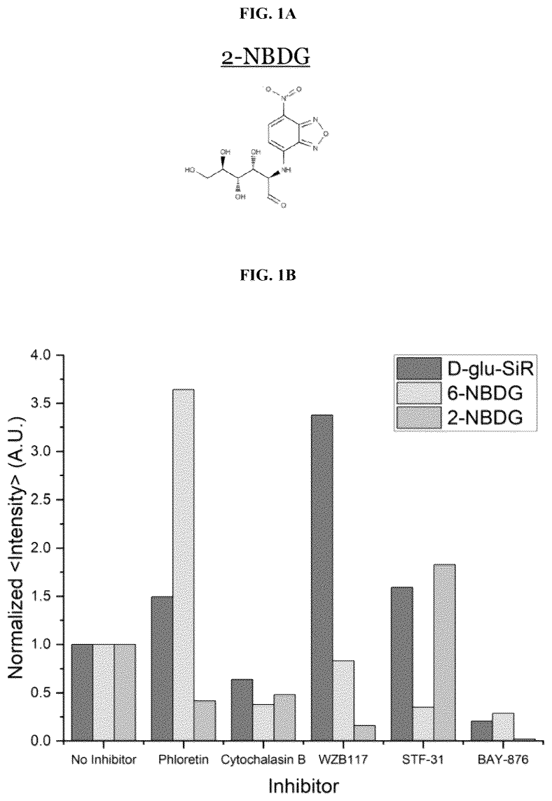

In certain embodiments, the method can further include contacting the anchored RBCs with a glucose analog. In certain embodiments, the glucose analog is a glucose analog that has affinity for the GLUT1 receptor on the surface of RBCs and internalized into the RBC by the GLUT1 receptor. In certain embodiments, the glucose analog is a non-hydrolyzable glucose analog. In certain embodiments, the glucose analog is a non-hydrolyzable glucose analog that is labeled, e.g., fluorescently labeled. In certain embodiments, the glucose analog comprises a fluorescent label. In certain embodiments, the fluorescently labeled glucose analog is a glucose analog coupled to a fluorophore. In certain embodiments, glucose analogs for use in the present disclosure include labeled deoxyglucose analogs, e.g., fluorescently labeled deoxyglucose analogs. Non-limiting examples include 6FGA (C6-fluorphore-deoxy-D-glucose analog), 2-DG-750 (2-deoxyglucosone 750), 2-NBDG (2-(N-(7-Nitrobenz-2-oxa-1,3-diazol-4-yl)amino)-2-deoxyglucose), 6-NBDG (6-(N-(7-Nitrobenz-2-oxa-1,3-diazol-4-yl)amino)-2-deoxyglucose), GB2-Cy3, GB2-Cy5, Cy3-glucosamine, Cy5-glucosamine and D-glucose-Silicon rhodamine (D-glucose-SiR). In certain embodiments, the glucose analog is 2-NBDG. As shown in FIG. 1, 2-NBDG is primarily internalized via the GLUT1 receptor compared to the other tested glucose analogs.

In certain embodiments, the RBCs anchored to the substrate are contacted with the glucose analog at a concentration from about 0.5 mM to about 50 mM. For example, but not by way of limitation, the RBCs anchored to the substrate are contacted with the glucose analog at a concentration from about 1 mM to about 45 mM, about 1 mM to about 40 mM, about 1 mM to about 35 mM, about 1 mM to about 30 mM, about 1 mM to about 25 mM, about 1 mM to about 20 mM, about 1 mM to about 15 mM, about 1 mM to about 10 mM, about 1 mM to about 5 mM, about 5 mM to about 50 mM, about 10 mM to about 50 mM, about 15 mM to about 50 mM, about 20 mM to about 50 mM, about 25 mM to about 50 mM, about 30 mM to about 50 mM, about 35 mM to about 50 mM, about 40 mM to about 50 mM, about 45 mM to about 50 mM, about 5 mM to about 40 mM, about 10 mM to about 30 mM, about 5 mM to about 30 mM, about 5 mM to about 20 mM or about 10 mM to about 20 mM. In certain embodiments, the anchored RBCs are contacted with the glucose analog at a concentration of about 0.5 mM to about 10 mM. In certain embodiments, the anchored RBCs are contacted with the glucose analog at a concentration of about 5 mM to about 30 mM. In certain embodiments, the anchored RBCs are contacted with the glucose analog at a concentration of about 20 mM. In certain embodiments, the anchored RBCs are contacted with the glucose analog at a concentration of about 10 mM. In certain embodiments, the anchored RBCs are contacted with the glucose analog at a concentration of about 5 mM.

In certain embodiments, the anchored RBCs are contacted with the glucose analog at a concentration of about 1 mM to about 10 mM.

In certain embodiments, the anchored RBCs are contacted with the glucose analog at a concentration of about 1 mM to about 30 mM.

In certain embodiments, the anchored RBCs are contacted with the glucose analog at a concentration of about 1 mM to about 20 mM.

In certain embodiments, the method can further include imaging the internalization of the glucose analog by single RBCs. In certain embodiments, individual RBCs are imaged for a duration of about 1 to about 10 minutes, e.g., about 1 to about 9 minutes, about 1 to about 8 minutes, about 1 to about 7 minutes, about 1 to about 6 minutes, about 1 to about 5 minutes, about 1 to about 4 minutes or about 1 to about 3 minutes. In certain embodiments, individual RBCs are imaged for a duration of about 1 to about 6 minutes. In certain embodiments, sequential images of the individual RBCs can be obtained at a repetitive interval. For example, but not by way of limitation, sequential images of the individual RBCs can be obtained every second, every 2 seconds, every 3 second, every 4 seconds, every 5 seconds, every 6 seconds, every 7 seconds, every 8 seconds, every 9 seconds or every 10 seconds during a particular duration, e.g., a duration of about 1 to about 6 minutes. In certain embodiments, sequential images of an individual RBC can be obtained every second for a period of about 1 to about 10 minutes, e.g., about 3 minutes, to obtain a time trace of the glucose analog as it is internalized. In certain embodiments, sequential images of an individual RBC can be obtained every 2 seconds for a period of about 1 to about 10 minutes, e.g., about 3 minutes, to obtain a time trace of the glucose analog as it is internalized. In certain embodiments, about 10 to about 500 sequential images can be obtained during a time period described herein, e.g., about 10 to about 400, about 10 to about 300, about 10 to about 200, about 10 to about 100 or about 10 to about 50 sequential images can be obtained during the perfusion of the glucose analog over the anchored RBCs.

In certain embodiments, a subset of the population of RBCs can be imaged together. For example, but not by way of limitation, a subset of the population of RBCs can be imaged simultaneously. In certain embodiments, at least about 2 of RBCs imaged together, e.g., at least about 5, at least about 10, at least about 50, at least about 100, at least about 150, at least about 200, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900 or at least about 1,000. In certain embodiments, at least about 10 of RBCs imaged together. In certain embodiments, at least about 50 of RBCs imaged together. In certain embodiments, at least about 100 of RBCs imaged together. In certain embodiments, at least about 200 of RBCs imaged together. In certain embodiments, at least about 300 of RBCs imaged together. In certain embodiments, at least about 400 of RBCs imaged together. In certain embodiments, at least about 500 of RBCs imaged together. In certain embodiments, at least about 750 of RBCs imaged together. In certain embodiments, the number of RBCs imaged together can be from about 1 to about 1,000, from about 1 to about 950, from about 1 to about 900, from about 1 to about 850, from about 1 to about 800, from about 1 to about 750, from about 1 to about 700, from about 1 to about 650, from about 1 to about 600, from about 1 to about 550, from about 1 to about 500, from about 1 to about 450, from about 1 to about 400, from about 1 to about 350, from about 1 to about 300, from about 1 to about 250, from about 1 to about 200, from about 1 to about 150, from about 1 to about 100, from about 1 to about 50, from about 1 to about 10, from about 5 to about 1,000, from about 10 to about 1,000, from about 50 to about 1,000, from about 100 to about 1,000, from about 150 to about 1,000, from about 200 to about 1,000, from about 250 to about 1,000, from about 300 to about 1,000, from about 350 to about 1,000, from about 400 to about 1,000, from about 450 to about 1,000, from about 500 to about 1,000, from about 550 to about 1,000, from about 600 to about 1,000, from about 650 to about 1,000, from about 700 to about 1,000, from about 750 to about 1,000, from about 800 to about 1,000, from about 850 to about 1,000, from about 900 to about 1,000, from about 950 to about 1,000, from about 50 to about 900, from about 100 to about 800, from about 200 to about 700, from about 200 to about 600, from about 200 to about 500, from about 200 to about 400 or from about 300 to about 400. In certain embodiments, the number of RBCs imaged together can be from about 1 to about 400. In certain embodiments, the number of RBCs imaged together can be from about 1 to about 300. In certain embodiments, the number of RBCs imaged together can be from about 100 to about 400. In certain embodiments, the number of RBCs imaged together can be from about 100 to about 300.

In certain embodiments, the number of RBCs imaged together can be from about 300 to about 400.

In certain embodiments, the RBCs are imaged under physiological conditions, e.g., conditions that mimic in vivo conditions. In certain embodiments, the RBCs are imaged under physiological conditions that mimic the flow of RBCs in the blood through the vasculature, e.g., including physiological temperature and flow rate. In certain embodiments, the RBCs are imaged in the presence of a flow rate that mimics venous pressure. In certain embodiments, the RBCs are imaged in the presence of a flow rate that mimics venous pressure while avoiding shearing of the RBCs. In certain embodiments, the flow rate can be from about 7.5 to about 55 μL/hr, e.g., from about 7.5 to about 20 μL/hr, about 7.5 to about 30 μL/hr, about 7.5 to about 40 μL/hr, about 7.5 to about 50 μL/hr, about 10 to about 55 μL/hr, about 20 to about 55 μL/hr, about 30 to about 55 μL/hr, about 40 to about 55 μL/hr, about 50 to about 55 μL/hr, about 20 to about 40 μL/hr, about 10 to about 20 μL/hr, about 10 to about 30 μL/hr, about 10 to about 40 μL/hr or about 20 to about 30 μL/hr.

In certain embodiments, the RBCs are imaged in the presence of a flow rate from about 10 to about 30 μL/hr.

In certain embodiments, the anchored RBCs are contacted with the glucose analog by perfusion, e.g., the solution comprising the glucose analog is flowed across the anchored RBCs at a perfusion rate that allows the internalization of the glucose analog by the RBCs. In certain embodiments, the perfusion rate is from about 1 μL/hr to about 500 μL/hr, e.g., about 1 μL/hr to about 400 μL/hr, about 1 μL/hr to about 300 μL/hr, about 1 μL/hr to about 200 μL/hr, about 1 μL/hr to about 100 μL/hr, about 1 μL/hr to about 50 μL/hr, about 1 μL/hr to about 40 μL/hr, about 1 μL/hr to about 30 μL/hr, about 5 μl/hr to about 100 μL/hr, about 10 μL/hr to about 100 μL/hr, about 10 μL/hr to about 50 μL/hr or about 10 μL/hr to about 30 μL/hr. In certain embodiments, the perfusion rate is from about 1 μL/hr to about 50 μL/hr.

In certain embodiments, the anchored RBCs are contacted with the glucose analog at a perfusion rate from about 1 μL/hr to about 30 μL/hr.

In certain embodiments, the anchored RBCs are contacted with the glucose analog at a perfusion rate from about 10 μL/hr to about 30 μL/hr.

In certain embodiments, the anchored RBCs are contacted with the glucose analog at a perfusion rate of about 20 μL/hr.

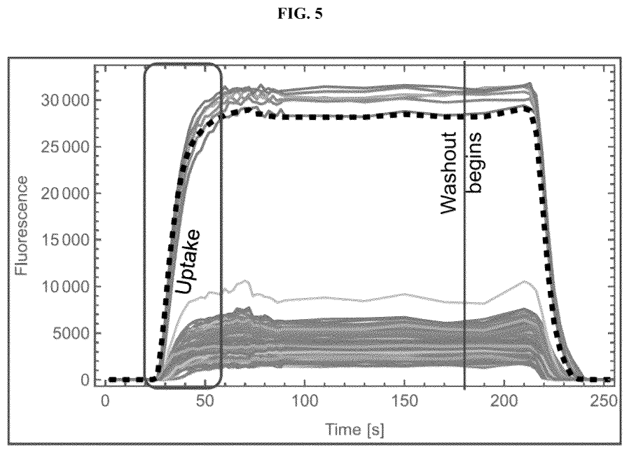

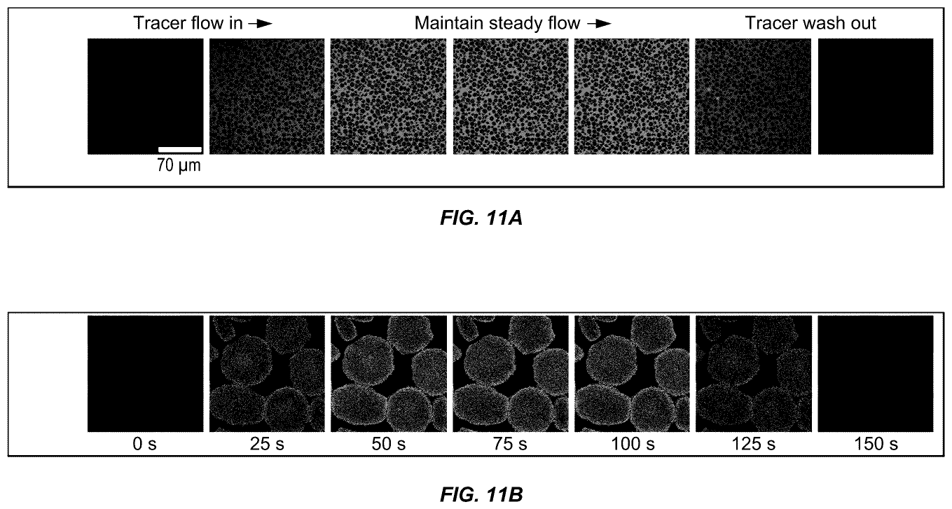

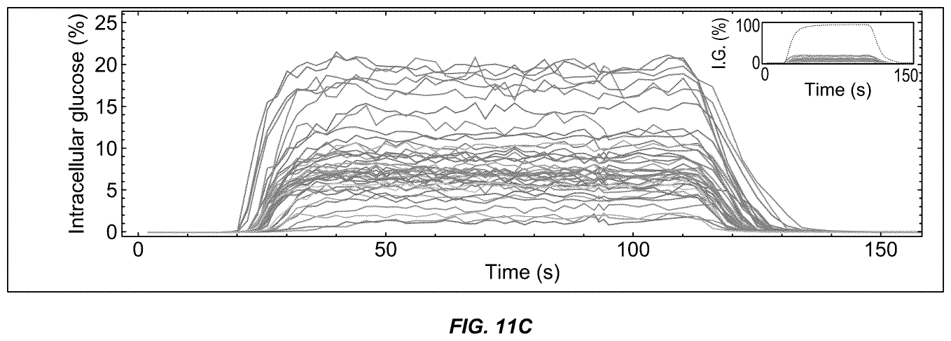

In certain embodiments, the method can further include washing out the glucose analog. The glucose analog can be washed out with a solution that does not include the glucose analog. In certain embodiments, images can be obtained of the RBCs during this washout period, e.g., as shown in FIG. 5 and FIGS. 11A-11B. In certain embodiments, the solution is a buffer. In certain embodiments, the solution is a buffer that does not include the glucose analog. In certain embodiments, the solution is a buffer to maintain osmolarity and/or prevent rupture and/or lysis of the RBCs during imaging. In certain embodiments, the buffer includes one or more of the following components: a zwitterionic compound (e.g., HEPES), EGTA and one or more salts (e.g., NaCl and/or KCl). In certain embodiments, the buffer includes from about 90 mM to about 150 mM of at least one salt (e.g., KCl), e.g., about 100 mM of at least one salt (e.g., KCl) or 125 mM of at least one salt (e.g., KCl). In certain embodiments, the buffer includes from about 1 mM to about 10 mM of a zwitterionic compound, e.g., 5 mM of a zwitterionic compound. In certain embodiments, the buffer includes from about 1 mM to about 10 mM of HEPES, e.g., 5 mM of HEPES. In certain embodiments, the buffer includes from about 1 mM to about 10 mM of EGTA, e.g., 4 mM of EGTA. In certain embodiments, the buffer includes from about 1 mM to about 10 mM of NaCl, e.g., 5 mM of NaCl. In certain embodiments, the buffer has a pH from about 7 to about 8, e.g., a pH of about 7.4. In certain embodiments, the buffer includes from about 1 mM to about 200 mM of at least one salt, from about 1 mM to about 10 mM of a zwitterionic compound and from about 1 mM to about 10 mM of EGTA (e.g., 4 mM of EGTA) and has a pH from about 7 to about 8 (e.g., a pH of about 7.4). In certain embodiments, the buffer includes from about 90 mM to about 150 mM of KCl (e.g., about 100 mM of KCl or 125 mM KCl), from about 1 mM to about 10 mM of HEPES (e.g., 5 mM of HEPES), from about 1 mM to about 10 mM of EGTA (e.g., 4 mM of EGTA), from about 1 mM to about 10 mM of NaCl (e.g., 5 mM of NaCl) and has a pH from about 7 to about 8 (e.g., a pH of about 7.4).

In certain embodiments, individual RBCs are imaged for a duration of about 1 to about 10 minutes, e.g., about 1 to about 9 minutes, about 1 to about 8 minutes, about 1 to about 7 minutes, about 1 to about 6 minutes, about 1 to about 5 minutes, about 1 to about 4 minutes or about 1 to about 3 minutes, during the washout period. In certain embodiments, individual RBCs are imaged for a duration of about 1 to about 6 minutes during the washout period. In certain embodiments, sequential images of the individual RBCs can be obtained at a repetitive interval during the washout period. For example, but not by way of limitation, sequential images of the individual RBCs can be obtained every second, every 2 seconds, every 3 second, every 4 seconds, every 5 seconds, every 6 seconds, every 7 seconds, every 8 seconds, every 9 seconds or every 10 seconds during a particular duration, e.g., a duration of about 1 to about 6 minutes, during the washout period. In certain embodiments, sequential images of an individual RBC can be obtained every second for a period of about 1 to about 10 minutes, e.g., about 3 minutes, during the washout period. In certain embodiments, sequential images of an individual RBC can be obtained every 2 seconds for a period of about 1 to about 10 minutes, e.g., about 3 minutes, during the washout period. In certain embodiments, sequential images of an individual RBC can be obtained every 3 seconds for a period of about 1 to about 10 minutes, e.g., about 3 minutes, during the washout period. In certain embodiments, about 10 to about 500 sequential images can be obtained during the washout period, e.g., about 10 to about 400, about 10 to about 300, about 10 to about 200, about 10 to about 100 or about 10 to about 50 sequential images can be obtained during the washout period.

In certain embodiments, methods of the present disclosure can further include obtaining images along the z-axis of the RBCs, e.g., along the height of the RBCs, e.g., perpendicular to the substrate. In certain embodiments, the set of images obtained along the z-axis at a timepoint (or during a short timeframe) is referred to as a z-stack. For example, but not by way of limitation, images along the z-axis can be obtained at least once during the flow of the glucose analog over the anchored RBCs, obtained at least once during the “plateau” stage during the flow of the glucose analog over the anchored RBCs (e.g., the full concentration flow of the glucose analog over the anchored RBCs) and/or obtained at least once during the washout period. In certain embodiments, images along the z-axis can be obtained at least once during the flow of the glucose analog over the anchored RBCs. In certain embodiments, images along the z-axis can be obtained at least once during the “plateau” stage during the flow of the glucose analog over the anchored RBCs. In certain embodiments, images along the z-axis can be obtained at least once during the washout period. In certain embodiments, images along the z-axis are obtained at least once during the flow of the glucose analog over the anchored RBCs, obtained at least once during the “plateau” stage during the flow of the glucose analog over the anchored RBCs (e.g., the full concentration flow of the glucose analog over the anchored RBCs) and obtained at least once during the washout period. In certain embodiments, at least about 10 to about 500 images along the z-axis can be obtained, e.g., about 10 to about 400, about 10 to about 300, about 10 to about 200, about 10 to about 100 or about 10 to about 70 images can be obtained along the z-axis, at a step size of about 0.01 μm to about 5 μm, e.g., a step size of about 0.1 μm.

In certain embodiments, the anchored RBCs are contacted with different concentrations of the glucose analog at different time points. For example, but not by way of limitation, the anchored RBCs can be contacted with increasing concentrations of the glucose analog. In certain embodiments, the anchored RBCs can be contacted with a first concentration of the glucose analog followed by contacting the anchored RBCs with a second concentration of the glucose analog, e.g., where the first concentration of the glucose analog is washed out prior to contacting the anchored RBCs with the second concentration of glucose analog. In certain embodiments, the anchored RBCs can be contacted with at least two different concentrations of the glucose analog, e.g., in any order or in order of increasing concentration. In certain embodiments, the anchored RBCs can be contacted with at least three different concentrations of the glucose analog, e.g., in any order or in order of increasing concentration. In certain embodiments, the anchored RBCs can be contacted with at least four different concentrations of the glucose analog, e.g., in any order or in order of increasing concentration. In certain embodiments, the anchored RBCs can be contacted with at least five different concentrations of the glucose analog, e.g., in any order or in order of increasing concentration. In certain embodiments, the concentration of the glucose analog increases in increments of about 1 mM, of about 5 mM or of about 10 mM. In certain embodiments, the concentration of the glucose analog increases in increments of about 5 mM. In certain embodiments, the concentration of the glucose analog increases in increments of about 10 mM. In certain embodiments, the previous concentration of glucose analog can be washed out, e.g., using a solution that does not include a glucose analog as described above, before contacting the anchored RBCs with the glucose analog at a different concentration.

In certain embodiments, the anchored RBCs are contacted with the glucose analog at a concentration of about 1 mM, about 5 mM, of about 10 mM, of about 15 mM, of about 20 mM, of about 25 mM and/or of about 30 mM. In certain embodiments, the anchored RBCs are contacted with the glucose analog at a concentration of about 1 mM, of about 5 mM and of about 10 mM. In certain embodiments, the anchored RBCs are contacted with the glucose analog at a concentration of about 5 mM, of about 10 mM, of about 20 mM and of about 30 mM. In certain embodiments, the anchored RBCs are contacted with the glucose analog at a concentration of about 1 mM, of about 5 mM, of about 10 mM, of about 15 mM, of about 20 mM, of about 25 mM and of about 30 mM. By anchoring the RBCs to the substrate, the same RBCs (e.g., at the same spatial location on the substrate) can be analyzed in the presence of increasing concentrations of the glucose analog, which allows for a greater understanding of the internalization of glucose in individual RBCs.

In certain embodiments, the method can further include analyzing the kinetics of glucose internalization for each individual RBC imaged. In certain embodiments, the method can further include analyzing the kinetics of glucose analog internalization for each individual RBC imaged at a specific concentration of the glucose analog. For example, but not by way of limitation, the method of the present disclosure can include analyzing the kinetics of glucose analog internalization for each individual RBC imaged (i) to determine the level (e.g., maximum level) of intracellular glucose analog in each RBC, (ii) to determine the internalization rate of the glucose analog in each RBC and/or (iii) to determine the ratio of the intracellular level to the extracellular level of the glucose analog of each RBC. In certain embodiments, the method of the present disclosure can include analyzing the kinetics of glucose analog internalization for each individual RBC imaged to determine the level (e.g., maximum level) of intracellular glucose analog in each RBC. In certain embodiments, the method of the present disclosure can include analyzing the kinetics of glucose analog internalization for each individual RBC imaged to determine the internalization rate of the glucose analog in each RBC. In certain embodiments, the method of the present disclosure can include analyzing the kinetics of glucose analog internalization for each individual RBC imaged to determine the ratio of the intracellular level to the extracellular level of the glucose analog of each RBC.

In certain embodiments, the method of the present disclosure can include analyzing the kinetics of glucose analog internalization for each individual RBC imaged at single concentrations or multiple concentrations of the glucose analog (i) to determine the intracellular level (e.g., maximum level) of the glucose analog in each RBC at a single concentration or multiple concentrations of the glucose analog, (ii) to determine the internalization rate of the glucose analog in each RBC at a single concentration or multiple concentrations of the glucose analog and/or (iii) to determine the ratio of the amount of internalized to the amount extracellular glucose analog of each RBC at a single concentration or multiple concentrations of the glucose analog. In certain embodiments, the method of the present disclosure can include analyzing the kinetics of glucose analog internalization for each individual RBC imaged at single concentrations or multiple concentrations of the glucose analog to determine the intracellular level (e.g., maximum level) of the glucose analog in each RBC at a single concentration or multiple concentrations of the glucose analog. In certain embodiments, the method of the present disclosure can include analyzing the kinetics of glucose analog internalization for each individual RBC imaged at single concentrations or multiple concentrations of the glucose analog to determine the internalization rate of the glucose analog in each RBC at a single concentration or multiple concentrations of the glucose analog. In certain embodiments, the method of the present disclosure can include analyzing the kinetics of glucose analog internalization for each individual RBC imaged at single concentrations or multiple concentrations of the glucose analog to determine the ratio of the amount of internalized to the amount extracellular glucose analog of each RBC at a single concentration or multiple concentrations of the glucose analog.

In certain embodiments, the method can further include analyzing the kinetics of glucose internalization for each individual RBC imaged to determine the kinetics of glucose internalization for the population of RBCs imaged or for a subset of the population of RBCs imaged. For example, but not by way of limitation, the method of the present disclosure can include analyzing the kinetics of glucose analog internalization for each individual RBC imaged (i) to determine the intracellular level (e.g., maximum level) of the glucose analog of a population of RBCs (e.g., or a subset of the population), (ii) to determine the internalization rate of the glucose analog of a population of RBCs (e.g., or a subset of the population) and/or (iii) to determine the ratio of the intracellular level to the extracellular level of the glucose analog among a population of RBCs (e.g., or a subset of the population). In certain embodiments, the method can include analyzing the kinetics of glucose internalization for each individual RBC imaged to determine the intracellular level (e.g., maximum level) of the glucose analog of a population of RBCs (e.g., or a subset of the population). In certain embodiments, the method can include analyzing the kinetics of glucose internalization for each individual RBC imaged to determine the internalization rate of the glucose analog of a population of RBCs (e.g., or a subset of the population). In certain embodiments, the method can include analyzing the kinetics of glucose internalization for each individual RBC imaged to determine the ratio of the intracellular level to the extracellular level of the glucose analog among a population of RBCs (e.g., or a subset of the population). In certain embodiments, the population of RBCs is obtained from a single patient.

In certain embodiments, the method of the present disclosure can include analyzing the kinetics of glucose analog internalization for each individual RBC imaged at single concentrations or multiple concentrations of the glucose analog (i) to determine the intracellular level (e.g., maximum level) of the glucose analog for the population of RBCs imaged (e.g., or a subset of the population) at a single concentration or multiple concentrations of the glucose analog, (ii) to determine the internalization rate of the glucose analog for the population of RBCs imaged (e.g., or a subset of the population) at a single concentration or multiple concentrations of the glucose analog and/or (iii) to determine the ratio of the intracellular level to the extracellular level of the glucose analog for the population of RBCs imaged (e.g., or a subset of the population) at a single concentration or multiple concentrations of the glucose analog. In certain embodiments, the method of the present disclosure can include analyzing the kinetics of glucose analog internalization to determine the intracellular level (e.g., maximum level) of the glucose analog for the population of RBCs imaged (e.g., or a subset of the population) at a single concentration or multiple concentrations of the glucose analog. In certain embodiments, the method of the present disclosure can include analyzing the kinetics of glucose analog internalization to determine the internalization rate of the glucose analog for the population of RBCs imaged (e.g., or a subset of the population) at a single concentration or multiple concentrations of the glucose analog. In certain embodiments, the method of the present disclosure can include analyzing the kinetics of glucose analog internalization to determine the ratio of the intracellular level to the extracellular level of the glucose analog for the population of RBCs imaged (e.g., or a subset of the population) at a single concentration or multiple concentrations of the glucose analog. In certain embodiments, the population of RBCs is obtained from a single patient.

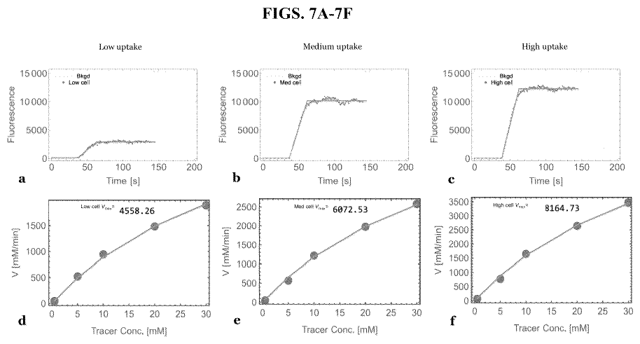

In certain embodiments, the method can include determining the intracellular level (e.g., maximum level) of the glucose analog in the single RBCs, e.g., at a single concentration or at multiple concentrations of the glucose analog. In certain embodiments, the method includes determining the maximum intracellular level of the glucose analog. For example, but not by way of limitation, the intracellular levels (e.g., maximum level) of the glucose analog in single RBCs can be determined by calculating the amount of glucose analog at the plateau of a graph that plots fluorescence versus time as shown in FIG. 5 and FIG. 7. In certain embodiments, the method can include determining the average intracellular level of the glucose analog in a population of RBCs, e.g., at a single concentration or at multiple concentrations of the glucose analog. In certain embodiments, the method can include determining the median intracellular level of the glucose analog in a population of RBCs, e.g., at a single concentration or at multiple concentrations of the glucose analog. In certain embodiments, the population of RBCs is obtained from a single patient.

In certain embodiments, the method can include determining the uptake rate of the glucose tracer in single RBCs, e.g., at a single concentration or at multiple concentrations of the glucose analog. In certain embodiments, the method can include determining the average uptake rate of a population of RBCs, e.g., at a single concentration or at multiple concentrations of the glucose analog. In certain embodiments, the method can include determining the median uptake rate of a population of RBCs, e.g., at a single concentration or at multiple concentrations of the glucose analog. In certain embodiments, the population of RBCs is obtained from a single patient.

In certain embodiments, the method can include determining the Vₘₐₓ of single RBCs, e.g., at a single concentration or at multiple concentrations of the glucose analog. In certain embodiments, the method can include determining the average Vₘₐₓ of a population of RBCs, e.g., at a single concentration or at multiple concentrations of the glucose analog, as shown in FIG. 7. In certain embodiments, the method can include determining the median Vₘₐₓ of a population of RBCs, e.g., at a single concentration or at multiple concentrations of the glucose analog. In certain embodiments, the population of RBCs is obtained from a single patient.

In certain embodiments, the method can include determining the ratio of the intracellular level to the extracellular level of the glucose analog of single RBCs, e.g., at a single concentration or at multiple concentrations of the glucose analog. In certain embodiments, the method can include determining the average ratio of the intracellular level to the extracellular level of the glucose analog for a population of RBCs, e.g., at a single concentration or at multiple concentrations of the glucose analog, as shown in FIG. 8. In certain embodiments, the method can include determining the median ratio of the intracellular level to the extracellular level of the glucose analog for a population of RBCS, e.g., at a single concentration or at multiple concentrations of the glucose analog. In certain embodiments, the population of RBCs is obtained from a single patient.

In certain embodiments, microfluidic systems can be used to perform the methods of the present disclosure. A microfluidic system generally includes any system in which very small volumes of fluid are stored and manipulated. In certain embodiments, microfluidic systems carry fluid in predefined paths through one or more microfluidic passages, which can have a minimum dimension, e.g., height or width. In certain embodiments, microfluidic systems include one or more sets of passages that interconnect to form a generally closed microfluidic network. In certain embodiments, the microfluidic network can include one, two, three, four or more openings that interface with the outside environment and can receive, store and/or dispense fluid. In certain embodiments, fluid can be dispensed directly into the microfluidic network or sites external the microfluidic system to allow contact of the fluid with the cells, e.g., RBCs, in the cell culture chambers of the microfluidic system. In certain embodiments, the microfluidic system can include regulatory or control mechanisms that determine aspects of fluid flow rate and/or path. In certain embodiments, the microfluidic system can also include mechanisms that determine, regulate, and/or sense fluid temperature, fluid pressure and/or fluid flow rate. Microfluidic systems for use in the present disclosure can be formed of any suitable material including, but not limited to, polydimethylsiloxane (PDMS), polystyrene, polypropylene, polycarbonate, glass, ceramics, silicon and metals. In certain embodiments, the microfluidic system for use in the present disclosure is the CellASIC ONIX perfusion system.

### III. Clinical Uses

In certain embodiments, the information obtained by the methods of the present disclosure can be used to inform a more personalized diagnostic or therapeutic strategy for pre-diabetic or diabetic patients. For example, but not by way of limitation, information obtained from the methods of the present disclosure can be used to overcome the limitations of laboratory HbA1c as described herein. In certain embodiments, information from the methods of the present disclosure can be used to predict, modify, evaluate, correct and/or adjust a patient's HbA1c.

In certain embodiments, a patient's HbA1c can be measured in a laboratory and/or calculated based, at least in part, on glucose monitoring data. In certain embodiments, the glucose monitoring data is continuous with little to no missed readings to provide higher accuracy in the calculated HbA1c level. Several methods can be used for calculating (or estimating) an HbA1c level including, but not limited to, the cAG/A1C Conversion Calculator provided by the American Diabetes Association; glucose management indicator (GMI) methods (e.g., methods described in Glucose management indicator (GMI): A new term for estimating AIC from continuous glucose monitoring Diabetes 41(11): 2275-2280 November 2018); methods described in Translating the AIC assay into estimated average glucose values Diabetes Care 31(8): 1473-8 Aug. 2008 PMID: 18540046; methods described in Mechanistic modeling of hemoglobin glycation and red blood cell kinetics enables personalized diabetes monitoring Sci. Transl. Med. 8, 359ral30 Oct. 2016; US Pat. App. Pub. No. 2018/0235524; U.S. Prov. Pat. App. No. 62/750,957; and U.S. Prov. Pat. App. No. 62/939,956. Each of the foregoing patent applications are incorporated herein by reference in their entirety for all purposes. In certain embodiments, the patient's HbA1c is measured in a laboratory.

In certain embodiments, information obtained from the methods of the present disclosure can be used to predict, modify, evaluate, correct and/or adjust a patient's HbA1c as described in International Patent Publication WO 2021/108419 and U.S. patent application Ser. No. 18/052,805, the contents of each of which are incorporated herein by reference in their entirety for all purposes. For example, but not by way of limitation, the Vₘₐₓ values obtained from the methods of the present disclosure can be used in the Equation 3 of WO 2021/108419, replicated below.

$$[ GI ] = \frac{{V}_{max} * [ G ]}{{k}_{c} * ( {K}_{M} + [ G ] )} = \frac{{V}_{max}}{{K}_{M} * {k}_{c}} g \qquad Equation 3$$

In Equation 3 of WO 2021/108419, [GI] is the intracellular glucose concentration, g=(K_{M}*[G])/(K_{M}+[G]), k_{c} is the rate constant for glucose consumption in the red blood cell (e.g., having units of day-1) and K is the Michaelis-Menten kinetic rate constant for GLUT1 transporting glucose across the red blood cell membrane (e.g., having units of mM or mg/dL). In certain embodiments, K_{M} can have a value of 100 mg/dL to about 700 mg/dL, e.g., about 306 mg/dL or about 472 mg/dL. In certain embodiments, the average Vₘₐₓ value determined for a population of RBCs obtained from a patient can be used as the input for Vₘₐₓ of Equation 3 of WO 2021/108419. In certain embodiments, the median Vₘₐₓ value determined for a population of RBCs obtained from a patient can be used as the input for Vₘₐₓ of Equation 3 of WO 2021/108419.

In certain embodiments, the Vₘₐₓ values obtained from the methods of the present disclosure can be used to determine k_{gly} as disclosed in WO 2021/108419 (e.g., at paragraph [0039]). k_{gly} is the rate constant for glucose to be transported into an RBC and glycated to hemoglobin (Hb) to generate HbG. In certain embodiments, assuming a universal k_{c}, the average Vₘₐₓ value determined for a population of RBCs obtained from a patient can be used to estimate k_{gly} as disclosed in WO 2021/108419 (e.g., at paragraph) [0039]). In certain embodiments, the median Vₘₐₓ value determined for a population of RBCs obtained from a patient can be used to estimate k_{gly} as disclosed in WO 2021/108419 (e.g., at paragraph [0039]). For example, but not by way of limitation, k_{gly} can be determined by using the following equation: k_{gly}=k_{g}*Vₘₐₓ/(k_{c}*K_{M}), where k_{g} and K_{M} are universal constants for the non-enzymatic hemoglobin glycation reaction and glucose affinity to GLUT1, respectively.

In certain embodiments, information obtained from the methods of the present disclosure can be used to determine an adjusted HbA1c. In certain embodiments, an adjusted HbA1c can be determined using the equations provided in WO 2021/108419. In certain embodiments, an adjusted HbA1c can be determined using Equation 11 of WO 2021/108419 (e.g., paragraph of WO 2021/108419), replicated below.

$$aHbA 1 c = \frac{HbA 1 c}{HbA 1 c + \frac{K}{{K}^{ref}} ( 1 - HbA 1 c )} \qquad Equation 11$$

In Equation 11 of WO 2021/108419 above, HbA1c is the fraction of glycated hemoglobin molecules and can be cHbA1c or a laboratory HbA1c. In certain embodiments, the value of K^{ref} can be about 5.2×10⁻⁴ dL/mg. In certain embodiments, variable K of Equation 11 can be determined using Equation 5 disclosed WO 2021/108419 (e.g., paragraph of WO 2021/108419), which is as follows:

$$K = {k}_{gly} / {k}_{age} = [ HbG ] / ( g * [ Hb ] ) \qquad Equation 5$$

In Equation 5 of WO 2021/108419 above, [HbG] and [Hb] are the concentrations of glycated and un-glycated hemoglobin, respectively. g can be calculated as described above and k_{gly} of Equation 5 can be calculated as discussed above using Vₘₐₓ values obtained from the methods of the present disclosure. In certain embodiments, k_{age} can be determined by using the equation k_{age}=a*r/C, where C is the total hemoglobin concentration (e.g., C=[Hb]+[HbG]) and r is the red blood cell removal rate in units of concentration/time. a is a coefficient that has no units of measurement and is used to scale HbA1c to the fraction of glycated hemoglobin to be removed.

In certain embodiments, information obtained from the methods of the present disclosure can be used to predict a future HbA1c value of a patient. In certain embodiments, a future HbA1c value can be determined using the equations provided in WO 2021/108419. In certain embodiments, future HbA1c value can be determined using Equations 8 and 9 of WO 2021/108419 (e.g., paragraphs [0042]-[0044] of WO 2021/108419), which are replicated below.

$$HbA 1 {c}_{t} = EA + ( HbA 1 {c}_{0} - EA ) \cdot {e}^{- ( {k}_{gly} * g + {k}_{age} ) t} \qquad Equation 8$$

As disclosed in WO 2021/108419, under a hypothetical steady-state of constant glucose level, HbA1c should reach an equilibrium level, which is referred to as the “equilibrium HbA1c” (EA). HbA1c value HbA1Cₜ at the end of an interval t can be obtained from Equation 8, given a starting HbA1c (HbA1c₀) and assuming a constant glucose level during the time interval. In certain embodiments, variable k_{gly} of Equation 8 can be calculated as discussed above using Vₘₐₓ values obtained from the methods of the present disclosure. In certain embodiments, k_{age} can be determined as discussed above.

In certain embodiments, each patient's glucose history can be approximated as a series of time intervals tᵢ with corresponding glucose levels [Gᵢ] to accommodate changing glucose levels over time. In certain embodiments, a patient's HbA1c value at the end of time interval t_{z} (HbA1c_{z}) can be determined by Equation 9 of WO 2021/108419, replicated below.

$$HbA 1 {c}_{z} = {EA}_{z} ( 1 - {D}_{z} ) + {\sum }_{i = 1}^{z - 1} [ {EA}_{i} ( 1 - {D}_{i} ) {\prod }_{j = i + 1}^{z} {D}_{j} ] + HbA 1 {c}_{0} {\prod }_{j = 1}^{z} {D}_{j} \qquad Equation 9$$

In Equation 9 of WO 2021/108419 above, Dᵢ=e^{-(k}^{_{gly}}^{*g}^{ᵢ}^{+k}^{_{age}}^{)t}^{ᵢ}.

In certain embodiments, the intracellular level of the glucose analog in single RBCs can be used to inform a more personalized diagnostic or therapeutic strategy for pre-diabetic or diabetic patients. For example, but not by way of limitation, the intracellular level of the glucose analog in single RBCs obtained from a patient can be used to predict, modify, evaluate, correct and/or adjust the patient's HbA1c. In certain embodiments, the average intracellular level of the glucose analog in a population of RBCs obtained from a patient can be used to inform a diagnostic or therapeutic strategy for pre-diabetic or diabetic patients. In certain embodiments, the average intracellular level of the glucose analog in a population of RBCs obtained from a patient can be used to predict, modify, evaluate, correct and/or adjust the patient's HbA1c. In certain embodiments, the median intracellular level of the glucose analog in a population of RBCs obtained from a patient can be used to inform a more personalized diagnostic or therapeutic strategy for pre-diabetic or diabetic patients. In certain embodiments, the median intracellular level of the glucose analog in a population of RBCs obtained from a patient can be used to predict, modify, evaluate, correct and/or adjust the patient's HbA1c.

In certain embodiments, the uptake rate of the glucose analog in single RBCs can be used to inform a more personalized diagnostic or therapeutic strategy for pre-diabetic or diabetic patients. For example, but not by way of limitation, the uptake rate in single RBCs obtained from a patient can be used to predict, modify, evaluate, correct and/or adjust the patient's HbA1c. In certain embodiments, the average uptake rate in a population of RBCs obtained from a patient can be used to inform a more personalized diagnostic or therapeutic strategy for pre-diabetic or diabetic patients. In certain embodiments, the average uptake rate in a population of RBCs obtained from a patient can be used to predict, modify, evaluate, correct and/or adjust the patient's HbA1c. In certain embodiments, the median uptake rate in a population of RBCs obtained from a patient can be used to inform a more personalized diagnostic or therapeutic strategy for pre-diabetic or diabetic patients. In certain embodiments, the median uptake rate in a population of RBCs obtained from a patient can be used to predict, modify, evaluate, correct and/or adjust the patient's HbA1c.

In certain embodiments, the Vₘₐₓ in single RBCs can be used to inform a more personalized diagnostic or therapeutic strategy for pre-diabetic or diabetic patients. For example, but not by way of limitation, the Vₘₐₓ in single RBCs obtained from a patient can be used to predict, modify, evaluate, correct and/or adjust the patient's HbA1c. In certain embodiments, the average Vₘₐₓ in a population of RBCs obtained from a patient can be used to inform a more personalized diagnostic or therapeutic strategy for pre-diabetic or diabetic patients. In certain embodiments, the average Vₘₐₓ in a population of RBCs obtained from a patient can be used to predict, modify, evaluate, correct and/or adjust the patient's HbA1c. In certain embodiments, the median Vₘₐₓ in a population of RBCs obtained from a patient can be used to inform a more personalized diagnostic or therapeutic strategy for pre-diabetic or diabetic patients. In certain embodiments, the median Vₘₐₓ in a population of RBCs obtained from a patient can be used to predict, modify, evaluate, correct and/or adjust the patient's HbA1c.

In certain embodiments, the ratio of the intracellular level to the extracellular level of the glucose analog of single RBCs obtained from a patient can be used to inform a more personalized diagnostic or therapeutic strategy for pre-diabetic or diabetic patients. For example, but not by way of limitation, the ratio of the intracellular level to the extracellular level of the glucose analog of single RBCs obtained from a patient can be used to predict, modify, evaluate, correct and/or adjust the patient's HbA1c. In certain embodiments, the average ratio of the intracellular level to the extracellular level of the glucose analog in a population of RBCs obtained from a patient can be used to inform a more personalized diagnostic or therapeutic strategy for pre-diabetic or diabetic patients. In certain embodiments, the average ratio of the intracellular level to the extracellular level of the glucose analog in a population of RBCs obtained from a patient can be used to predict, modify, evaluate, correct and/or adjust the patient's HbA1c. In certain embodiments, the median ratio of the intracellular level to the extracellular level of the glucose analog in a population of RBCs obtained from a patient can be used to inform a more personalized diagnostic or therapeutic strategy for pre-diabetic or diabetic patients. In certain embodiments, the median ratio of the intracellular level to the extracellular level of the glucose analog in a population of RBCs obtained from a patient can be used to predict, modify, evaluate, correct and/or adjust the patient's HbA1c.

In certain embodiments, the information obtained by the methods of the present disclosure can be compared to reference values. For example, but not by way of limitation, the uptake rate of the glucose analog in single RBCs or in a population of RBCs from a single patient can be compared to a reference uptake rate. In certain embodiments, the Vₘₐₓ of single RBCs or the Vₘₐₓ of a population of RBCs from a single patient can be compared to a reference Vₘₐₓ. In certain embodiments, the ratio of the intracellular level to the extracellular level of the glucose analog of single RBCs or of a population of RBCs from a single patient can be compared to a reference ratio. In certain embodiments, the intracellular level of the glucose analog of single RBCs or of a population of RBCs from a single patient can be compared to a reference intracellular level of the glucose analog.

In certain embodiments, the reference value can be any reference value that is relevant to the patient from which the analyzed RBCs were obtained. In certain embodiments, the reference uptake rate can be the uptake rate, e.g., average or median uptake rate, of a plurality of subjects having at least one, e.g., at least two, demographic metrics in common with the patient. In certain embodiments, the reference ratio of the intracellular level to the extracellular level of the glucose analog can be the ratio, e.g., average or median ratio, of a plurality of subjects having at least one at least one, e.g., at least two, demographic metrics in common with the patient. In certain embodiments, the reference intracellular level of the glucose analog can be the intracellular level, e.g., average or median intracellular level, of a plurality of subjects having at least one, e.g., at least two, demographic metrics in common with the patient. In certain embodiments, the reference Vₘₐₓ can be the Vₘₐₓ, e.g., average or median Vₘₐₓ, of a plurality of subjects having at least one, e.g., at least two, demographic metrics in common with the patient. In certain embodiments, the demographic metric can include age, sex, race, pregnancy status or a combination thereof. In certain embodiments, the demographic metric can include age. In certain embodiments, the demographic metric can include pregnancy status. In certain embodiments, the demographic metric can include sex. In certain embodiments, the demographic metric can include race. As shown in Example 2, race has been observed to be a factor which contributes to differences in measured intracellular glucose fractions and such differences can be observed using the methods disclosed herein.

### IV. Kits

The presently disclosed subject matter further provides kits containing materials useful for performing the methods disclosed herein. In certain embodiments, a kit of the present disclosure includes a container containing one or more components of the cell anchoring system and/or a container containing one or more glucose analogs, described herein. Non-limiting examples of suitable containers include bottles, test tubes, vials and microtiter plates. The containers can be formed from a variety of materials such as glass or plastic.

In certain embodiments, the kit can include one or more containers containing one or more components of the cell anchoring system (e.g., as described in Section II above). In certain embodiments, the kit can include at least one container containing the antigen-binding agent coupled to biotin (e.g., as described in Section II above). For example, but not by way of limitation, the kit can include at least one container that includes one or more glucose analogs. In certain embodiments, the kit can include at least one container that includes 2-NBDG. In certain embodiments, a kit of the present disclosure can include a cell anchoring system (e.g., in a first container) and a glucose analog (e.g., in a second container).

In certain embodiments, the kit further includes a package insert that provides instructions for using the components provided in the kit. For example, a kit of the present disclosure can include a package insert that provides instructions for using the cell anchoring system components and/or the glucose analog of the kit in the disclosed methods. In certain embodiments, a kit of the present disclosure can include instructions for predicting, modifying, evaluating, correcting and/or adjusting a patient's HbA1c using the information obtained from the methods of the present disclosure. In certain embodiments, a kit of the present disclosure can include a cell anchoring system (e.g., in a first container), a glucose analog (e.g., in a second container) and instructions for using the cell anchoring system components and/or the glucose analog of the kit.

Alternatively or additionally, the kit can include other materials desirable from a commercial and user standpoint, including other buffers, diluents and filters. In certain embodiments, a kit of the present disclosure can comprise a container that includes a buffer, e.g., a homeostatic buffer described herein. In certain embodiments, the kit can include materials for collecting and/or processing blood samples, e.g., to isolate RBCs from a sample.

### V. Systems

The presently disclosed subject matter further provides systems useful for performing the methods disclosed herein. In certain embodiments, a system of the present disclosure includes a microfluidic device and/or a microscopy system, e.g., a confocal microscopy system. In certain embodiments, the microfluidic device includes a perfusion system.

In certain embodiments, the system can further include one or more containers, e.g., storage containers, containing one or more components of the cell anchoring system (e.g., as described in Section II above). In certain embodiments, the system can include at least one container, e.g., storage container, containing the antigen-binding agent coupled to biotin (e.g., as described in Section II above). For example, but not by way of limitation, the system can include at least one container, e.g., storage container, that includes one or more glucose analogs. In certain embodiments, the system can include at least one container, e.g., storage container, that includes 2-NBDG. In certain embodiments, a system of the present disclosure can include a cell anchoring system (e.g., in a first container), a glucose analog (e.g., in a second container) and a microfluidic device. In certain embodiments, a system of the present disclosure can include a cell anchoring system (e.g., in a first container), a glucose analog (e.g., in a second container), a microfluidic device and a microscopy system.

In certain embodiments, the system further includes documentation that provides instructions for using the system. For example, a system of the present disclosure can include a documentation that provides instructions for using the microfluidic device, the microscopy system, e.g., confocal microscopy system, the cell anchoring system components and/or the glucose analog of the system in the disclosed methods. In certain embodiments, a system of the present disclosure can include instructions for predicting, modifying, evaluating, correcting and/or adjusting a patient's HbA1c using the information obtained from the methods of the present disclosure. In certain embodiments, a system of the present disclosure can include a cell anchoring system (e.g., in a first container), a glucose analog (e.g., in a second container), a microfluidic device and instructions for using the microfluidic device, the cell anchoring system components and/or the glucose analog of the system. In certain embodiments, a system of the present disclosure can include a cell anchoring system (e.g., in a first container), a glucose analog (e.g., in a second container), a microfluidic device, a microscopy system and instructions for using the microfluidic device, the microscopy system, e.g., confocal microscopy system, the cell anchoring system components and/or the glucose analog of the system.

Alternatively or additionally, the system can include other materials desirable from a commercial and user standpoint, including other buffers, diluents and filters, e.g., in one or more additional containers, e.g., storage containers. In certain embodiments, a system of the present disclosure can comprise a container, e.g., storage container, that includes a buffer, e.g., a homeostatic buffer described herein.

### VI. Exemplary Embodiments

A. The presently disclosed subject matter provides a method for analyzing glucose internalization in single red blood cells, comprising:
(a) anchoring a population of red blood cells to a substrate to generate a population of anchored red blood cells;
(b) contacting the population of anchored red blood cells with a fluorescently labeled glucose analog;
(c) obtaining sequential images of the population of anchored red blood cells or a subset thereof of (b) for a length of time; and
(d) analyzing the internalization of the fluorescently labeled glucose analog in one or more single red blood cells of the population of anchored red blood cells or the subset thereof.

A.1. The presently disclosed subject matter provides a method for analyzing glucose internalization in single red blood cells, comprising:
(a) providing a population of red blood cells;
(b) anchoring the population of red blood cells to a substrate to generate a population of anchored red blood cells;
(c) contacting the population of anchored red blood cells with a fluorescently labeled glucose analog;
(d) obtaining sequential images of the population of anchored red blood cells or a subset thereof of (c) for a length of time; and
(e) analyzing the internalization of the fluorescently labeled glucose analogue in one or more single red blood cells of the population of anchored red blood cells or the subset thereof.

A.2. The presently disclosed subject matter provides a method for analyzing glucose internalization in single red blood cells, comprising:
(a) anchoring a population of red blood cells to a substrate to generate a population of anchored red blood cells;
(b) contacting the population of anchored red blood cells with a fluorescently labeled glucose analog;
(c) obtaining sequential images of the population of anchored red blood cells or a subset thereof of (b) for a length of time; and
(d) analyzing the internalization of the fluorescently labeled glucose analog in each red blood cell of the population of anchored red blood cells or the subset thereof.

A.3. The presently disclosed subject matter provides a method for analyzing glucose internalization in single red blood cells, comprising:
(a) providing a population of red blood cells;
(b) anchoring the population of red blood cells to a substrate to generate a population of anchored red blood cells;
(c) contacting the population of anchored red blood cells with a fluorescently labeled glucose analog;
(d) obtaining sequential images of the population of anchored red blood cells or a subset thereof of (c) for a length of time; and
(e) analyzing the internalization of the fluorescently labeled glucose analog in each red blood cell of the population of anchored red blood cells or the subset thereof.

A1. The method of any one of A-A.3, wherein the population of red blood cells is obtained from a single patient.

A1.1. The method of any one of A-A.3, wherein the population of red blood cells is obtained from a sample of a single patient.

A2. The method of A1 or A1.1, wherein the patient is nondiabetic, has prediabetes or has diabetes.

A3. The method of any one of A-A2, wherein the fluorescently labeled glucose analog is selected from the group consisting of 6FGA (C6-fluorphore-deoxy-D-glucose analog), 2-DG-750 (2-deoxyglucosone 750), 2-NBDG (2-(N-(7-Nitrobenz-2-oxa-1,3-diazol-4-yl)amino)-2-deoxyglucose), 6-NBDG (6-(N-(7-Nitrobenz-2-oxa-1,3-diazol-4-yl)amino)-2-deoxyglucose), GB2-Cy3, GB2-Cy5, Cy3-glucosamine, Cy5-glucosamine, D-glucose-Silicon rhodamine (D-glucose-SiR) and a combination thereof.

A3.1. The method of any one of A-A3, wherein the fluorescently labeled glucose analog is 2-NBDG.

A4. The method of any one of A-A3.1, wherein the fluorescently labeled glucose analog is contacted with the population of anchored red blood cells at a concentration from about 1 mM to about 30 mM.

A4.1. The method of A4, wherein the fluorescently labeled glucose analog is contacted with the population of anchored red blood cells at a concentration from about 1 mM to about 20 mM.

A4.2. The method of A4 or A4.1, wherein the fluorescently labeled glucose analog is contacted with the population of anchored red blood cells at a concentration from about 1 mM to about 10 mM.

A4.3. The method of any one of A4-A4.2, wherein the fluorescently labeled glucose analog is contacted with the population of anchored red blood cells at a concentration of about 5 mM.

A5. The method of any one of A-A4.3, wherein the population of anchored red blood cells are contacted with the fluorescently labeled glucose analog at more than one concentration, at more than two concentrations, at more than three concentrations, at more than four concentrations or at more than five concentrations.

A6. The method of any one of A-A5, wherein anchoring the population of red blood cells to the substrate comprises:
(a) coating the substrate with a serum albumin-biotin conjugate coupled to an avidin or an analog thereof;
(b) contacting the population of red blood cells with an antigen-binding molecule-biotin conjugate to generate a population of red blood cells coupled to the antigen-binding molecule-biotin conjugate; and
(c) contacting the population of red blood cells coupled to the antigen-binding molecule-biotin conjugate with the coated substrate of (a) to generate a population of anchored red blood cells.

A7. The method of A6, wherein the antigen-binding molecule-biotin conjugate binds to a protein on the surface of the red blood cells, wherein the protein is not a GLUT1 receptor.

A7.1. The method of A6 and A7, wherein the antigen-binding molecule of the antigen-binding molecule-biotin conjugate is an antibody.

A8. The method of A7 or A7.1, wherein the protein on the surface of the red blood cells is selected from the group consisting of Urea transporter (UT)-B, Aquaporin 1, Aquaporin 3, Rh-associated glycoprotein (CD241), CD59, CD47, Lutheran glycoprotein (CD329), Intercellular adhesion molecule-4 (ICAM4), ADP-ribosyltransferase 4 (CD297), Kell glycoprotein (CD238), Semaphorin 7A (CD108), Acetylcholinesterase, Erythroblast membrane-associated protein (ERMAP), Glycophorin A (CD235A), Glycophorin B (CD235B), Glycophorin C (CD236C), Glycophorin D (CD236D), Xg glycoprotein and a combination thereof.

A8.1. The method of any one of A7-A8, wherein the protein on the surface of the red blood cells is a glycophorin.

A8.2. The method of A8 and A8.1, wherein the glycophorin is Glycophorin A, Glycophorin B or Glycophorin A and Glycophorin B.

A9. The method of any one of A-A8.2, wherein the population of red blood cells comprises from about 5 to about 10,000 red blood cells.

A9.1. The method of any one of A-A9, wherein the population of RBCs comprises about 100 about 5,000 red blood cells.

A9.2. The method of any one of A-A9.1, wherein the population of RBCs comprises at least about 1,000 RBCs.

A9.3. The method of any one of A-A9.2 further comprising washing out the fluorescently labeled glucose analog.

A9.4. The method of A9.3, wherein the fluorescently labeled glucose analog is washed out with a solution that does not include the fluorescently labeled glucose analog.

A9.5. The method of A9.3 or A9.4 further comprising obtaining sequential images of the population of anchored red blood cells or a subset thereof of for a length of time during the washout period.

A10. The method of any one of A-A9.5, wherein (e) analyzing the internalization of the fluorescently labeled glucose analog in each single red blood cell of the population of anchored red blood cells or the subset thereof comprises determining an intracellular level of the glucose analog in each red blood cell.

A11. The method of A10 further comprising determining an average or median intracellular level of the glucose analog in the population of red blood cells or the subset thereof.

A12. The method of any one of A-A11, wherein (e) analyzing the internalization of the fluorescently labeled glucose analog in each single red blood cell of the population of anchored red blood cells or the subset thereof comprises determining a Vₘₐₓ for each red blood cell.

A13. The method of A12 further comprising determining an average or median Vₘₐₓ of the population of red blood cells or the subset thereof.

A14. The method of any one of A-A13, wherein (e) analyzing the internalization of the fluorescently labeled glucose analog in each single red blood cell of the population of anchored red blood cells or the subset thereof comprises determining a ratio of the intracellular level to the extracellular level of the glucose analog of each red blood cell.

A15. The method of A14 further comprising determining an average or median ratio of the intracellular level to the extracellular level of the glucose analog of the population of red blood cells or the subset thereof.

A16. The method of A10 or A11 further comprising comparing the intracellular level of the glucose analog in each red blood cell determined in A10 or the average or median intracellular level of the glucose analog determined in A11 to a reference intracellular level.

A17. The method of A12 or A13 further comprising comparing the Vₘₐₓ determined in A12 or the average or median Vₘₐₓ determined in A13 to a reference Vₘₐₓ.

A18. The method of A14 or A15 further comprising comparing the ratio determined in A14 or the average or median ratio determined in A15 to a reference ratio.

A19. The method of any one of A16-A18, wherein the reference intracellular level, the reference Vₘₐₓ and/or the reference ratio are values obtained from a plurality of subjects having at least one demographic metric in common with the patient.

A20. The method of A19, wherein the at least one demographic metric is race. A21. The method of any one of A10-A20, wherein the population of red blood cells comprises about 5 about 10,000 red blood cells.

A21.1. The method of any one of A10-A21, wherein the population of RBCs includes about 100 about 5,000 red blood cells.

A21.2. The method of any one of A10-A21.1, wherein the population of RBCs includes at least about 1,000 RBCs.

A22. The method of any one of A-A21.2, wherein the population of red blood cells has a red blood cell concentration from about 1×10² to about 1×10⁵ red blood cells/ml.

A23. The method of any one of A-A22, wherein the red blood cells of the population of red blood cells have a cell height greater than about 2.0 μm.

A24. The method of any one of A-A23, wherein anchoring of the red blood cells allows imaging of the same red blood cells for at least about 2 minutes, at least about 3 minutes, at least about 4 minutes or at least about 5 minutes at the same spatial location on the substrate.

A25. The method of any one of A6-A24, wherein the substrate is coated with the serum albumin-biotin conjugate by contacting the substrate with the serum albumin-biotin conjugate at a concentration from about 0.01 mg/ml to about 10 mg/ml.

A26. The method of any one of A6-A25, wherein the population of red blood cells are contacted with the antigen-binding molecule-biotin conjugate at a concentration of about 0.001 mg/ml to about 1 mg/ml.

A26.1. The method of any one of A6-A26, wherein the population of red blood cells are contacted with the antigen-binding molecule-biotin conjugate at a concentration of about 0.05 mg/ml to about 0.5 mg/ml.

A26.2. The method of any one of A6-A26.1, wherein the population of red blood cells are contacted with the antigen-binding molecule-biotin conjugate at a concentration of about 0.1 mg/ml.

A27. The method of any one of A-A26.2, wherein the population of red blood cells are contacted with the fluorescently labeled glucose analog at a concentration from about 0.5 mM to about 20 mM.

A28. The method of any one of A-A27, wherein the sequential images of the population of anchored red blood cells are obtained for a duration of about 1 to about 10 minutes in the presence of the glucose analog.

A29. The method of A28, wherein the sequential images of the population of anchored red blood cells are obtained every second or every 2 second for the duration.

A30. The method of any one of A-A29, wherein the sequential images of the population of anchored red blood cells are obtained for a duration of about 1 to about 10 minutes during the wash out of the fluorescently labeled glucose analog.

A31. The method of A30, wherein sequential images of the population of anchored red blood cells are obtained every second or every 2 second for the duration.

A32. The method of any one of A-A31, further comprising obtaining images along the z-axis of the red blood cells.

A33. The method of A32, wherein the images along the z-axis can be obtained at least once during the “plateau” stage.

A34. The method of any one of A-A33, wherein contacting the coated substrate with the population of red blood cells that were incubated with the antigen-binding molecule-biotin conjugate comprises flowing the population of red blood cells over the coated substrate at a perfusion rate from about 1 μL/hr to about 50 μL/hr.

A35. The method of A34, wherein the perfusion rate is from about 10 μL/hr to about 30 μL/hr.

A36. The method of A34 or A35, wherein the perfusion rate is about 20 μL/hr.

A37. The method of any one of A-A36, wherein the anchored red blood cells are contacted with the glucose analog at a perfusion rate that allows the internalization of the fluorescently labeled glucose analog by the red blood cells.

A38. The method of any one of A-A37, wherein the anchored red blood cells are contacted with the glucose analog at a perfusion rate of about 1 μL/hr to about 50 μL/hr.

A39. The method of A38, wherein the perfusion rate is from about 10 μL/hr to about 30 μL/hr.

A40. The method of A38 or A39, wherein the perfusion rate is about 20 μL/hr.

A41. The method of any one of A-A40, where the number of red blood cells imaged together (e.g., in a single field of view) can be from about 300 to about 400.

B. The presently disclosed subject matter provides a method of providing a personalized diagnosis and/or treatment to a patient using the intracellular level of the glucose analog of A10 or the average or median intracellular level of the glucose analog of claim A11.

B1. The presently disclosed subject matter provides a method of providing a personalized diagnosis to a patient using the intracellular level of the glucose analog of A10 or the average or median intracellular level of the glucose analog of claim A11.

B2. The presently disclosed subject matter provides a use of the intracellular level of the glucose analog of A10 or the average or median intracellular level of the glucose analog of claim A11 for treating a patient (e.g., personalized treatment of a patient).

C. The presently disclosed subject matter provides a method of providing a personalized diagnosis and/or treatment to a patient using the Vₘₐₓ of A12 or the average or median Vₘₐₓ of A13.

C1. The presently disclosed subject matter provides a method of providing a personalized diagnosis to a patient using the Vₘₐₓ of A12 or the average or median Vₘₐₓ of A13.

C2. The presently disclosed subject matter provides a use of the Vₘₐₓ of A12 or the average or median Vₘₐₓ of A13 for treating a patient (e.g., personalized treatment of a patient).

D. The presently disclosed subject matter provides a method of providing a personalized diagnosis and/or treatment to a patient using the ratio of A14 or the average or median ratio of A15.

D1. The presently disclosed subject matter provides a method of providing a personalized diagnosis to a patient using the ratio of A14 or the average or median ratio of A15.

D2. The presently disclosed subject matter provides a use of the ratio of A14 or the average or median ratio of A15 for treating a patient (e.g., personalized treatment of a patient).

E. Use of the intracellular level of the glucose analog of A10 or the average or median intracellular level of the glucose analog of claim A11 for providing a personalized diagnosis and/or for treating a patient.

E1. Use of the intracellular level of the glucose analog of A10 or the average or median intracellular level of the glucose analog of claim A11 for providing a personalized diagnosis of a patient.

E2. Use of the intracellular level of the glucose analog of A10 or the average or median intracellular level of the glucose analog of claim A11 for treating a patient (e.g., personalized treatment of a patient).

F. Use of the Vₘₐₓ of A12 or the average or median Vₘₐₓ of A13 for providing a personalized diagnosis and/or for treating a patient.

F1. Use of the Vₘₐₓ of A12 or the average or median Vₘₐₓ of A13 for providing a personalized diagnosis of a patient.

F2. Use of the Vₘₐₓ of A12 or the average or median Vₘₐₓ of A13 for treating a patient (e.g., personalized treatment of a patient).

G. Use of the Vₘₐₓ of A12 or the average or median Vₘₐₓ of A13 for adjusting predicting, modifying, evaluating, correcting and/or adjusting an HbA1c value of the patient.

H. Use of the ratio of A14 or the average or median ratio of A15 for providing a personalized diagnosis and/or for treating a patient.

H1. Use of the ratio of A14 or the average or median ratio of A15 for providing a personalized diagnosis of a patient.

H2. Use of the ratio of A14 or the average or median ratio of A15 for treating a patient (e.g., personalized treatment of a patient).

I. The presently disclosed subject matter provides a kit for performing the method or the use of any one of A-H2.

J. The presently disclosed subject matter provides a system for performing the method or the use of any one of A-H2.

### EXAMPLES

The following examples are merely illustrative of the presently disclosed subject matter and should not be considered as limiting in any way.

### Example 1: Glucose Uptake Assay

The study of glucose uptake in several different cell lines has been investigated for numerous purposes extending from understanding receptor kinetics to cellular pathologies. Human erythrocytes (red blood cells, RBCs) are a cell type where glucose uptake carries implications on human health (e.g., glycemia). Relevant to the diagnosis and treatment of diabetes mellitus, human RBCs are used in clinical testing to estimate a patient's time-averaged blood glucose level through measurement of glycated hemoglobin (HbA1c). Formation of HbA1c in RBCs is a function of duration of exposure and intracellular concentration of glucose. Historically, measurement of HbA1c and estimation of blood glucose has been performed through ensemble measurements on blood samples.

This example describes a method for determining the kinetics of glucose uptake in single RBCs. In particular, this example discloses the development of a fluorescence-based method to observe glucose uptake in individual RBCs. By incorporating microfluidics with confocal microscopy, single cell intracellular glucose percentage (as compared to the extracellular medium) were quantifiably determined.

In this example, 2-NBDG (2-(N-(7-Nitrobenz-2-oxa-1,3-diazol-4-yl)Amino)-2-deoxyglucose), which is a fluorescent glucose analog that consists of a glucosamine molecule with a substituted nitrobenzofurazan fluorophore at its amine group as shown in FIG. 1A, is used to visualize glucose uptake in single RBCs. As shown in FIG. 1B, flow cytometry experiments using human RBCs and various GLUT1 receptor inhibitors showed that 2-NBDG exhibited the most inhibition over other glucose tracers (D-glucose-SiR (D-glu-SiR) and 6-NBDG). Without being limited to a particular theory, these results suggest that the transport of 2-NBDG is mostly GLUT1-mediated. Further experiments were performed using 2-NBDG for monitoring glucose uptake in single RBCs.

To allow time-resolved measurements of glucose uptake in RBCs, the RBCs were anchored to a substrate for imaging using a cell anchoring system. A schematic of an exemplary cell anchoring system is shown in FIG. 2. As shown in FIG. 2, a substrate, e.g., a microscope glass coverslip (1), is coated with a bovine serum albumin (BSA)-biotin conjugate (2). Subsequently, a streptavidin-derivative (e.g., NeutrAvidin™) (3) interacts with the biotin of the BSA-biotin conjugate. RBCs (5) were incubated with an α-glycophorin-biotin conjugate (4) prior to placing them on the coverslip. Upon placing the RBCs on the coverslip, the other available binding sites of the streptavidin-derivative (e.g., NeutrAvidin™) molecule provides a strong and reliable method of attaching cells to the surface of the coverslip for imaging under flow conditions.

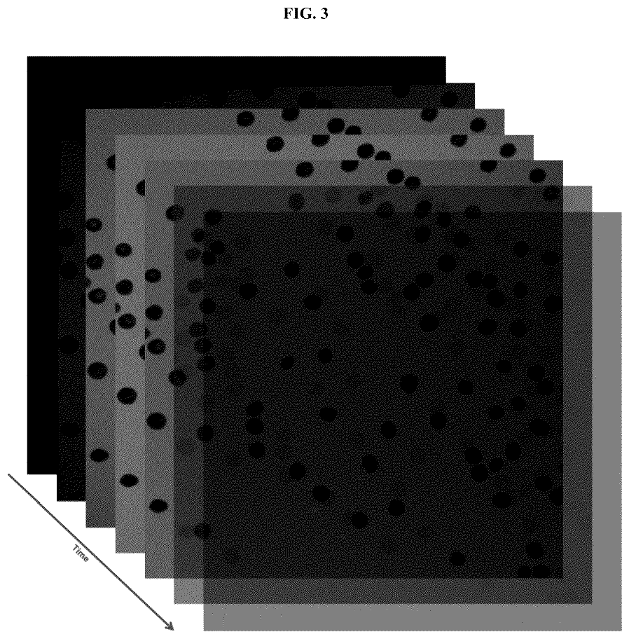

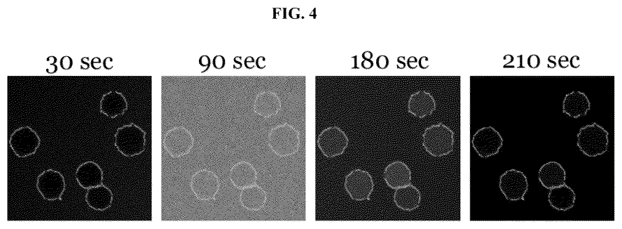

To image the RBCs, a commercially available Zeiss LSM980 system with a fully integrated package that is highly adaptable to many modes of imaging was used. In the current configuration, a CellASIC ONIX™ perfusion system was coupled to the Zeiss LSM980 system to perform flow-based experiments and reproduce conditions relevant for cell homeostasis and venous and arterial flow rates. A 488 nm laser was used for excitation of the 2-NBDG, and a Zeiss Zen controller was used to allow for filter selection of detection ranges of wavelengths. This prevented the signal from physiological concentrations of fluorescent tracer from saturating a detector. The microfluidic chambers provided 4 separate channels, but within each channel, there are 6 wells attached by channels which provided solution switching and homogeneous flow. The temperature controller unit also provided conditions even closer to that of physiological conditions. Incorporating the microfluidics perfusion system (CellASIC ONIX™) with a Zeiss Confocal system, allowed the generation of spatial intensity maps of cells throughout the course of introduction and wash out of the glucose tracer 2-NBDG as shown in FIG. 3. Using in-house written cell selection and isolation software, regions of interest (ROIs) were generated, which enclosed the RBCs and allowed for intensity measurements at each image during the time-resolved image sequence, as shown in FIG. 4.

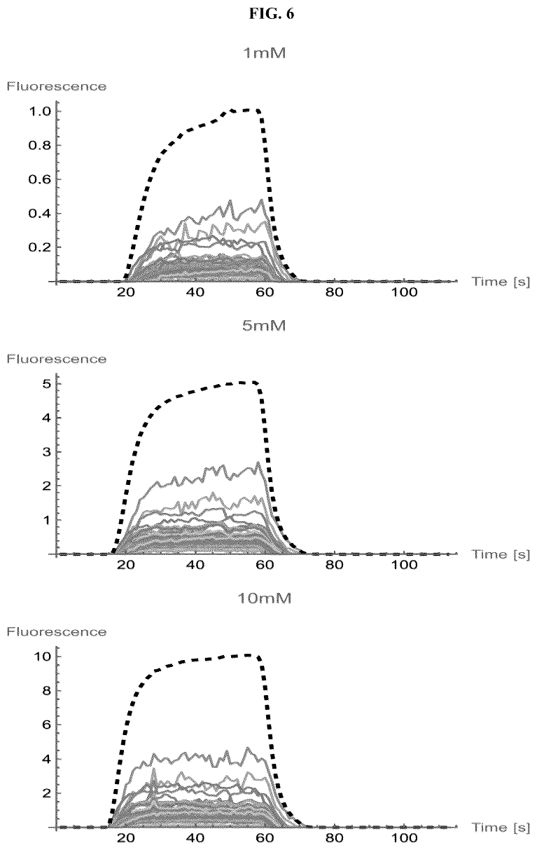

For each imaging field of view during a perfusion experiment, the number of ROIs can vary. However, each ROI provided full kinetic details of the glucose tracer uptake over a total of six minutes. Three minutes were used for perfusing the glucose tracer, 2-NBDG, while the remaining three minutes were used to flow only homeostasis buffer, which washed out the tracer both from the field of view and the cells. As shown in FIG. 5, the dashed line represents the median intensity of the extracellular glucose tracer (the background), and the other lines represent the spectrum of uptake in which different cells experience in the ROI during the experiment. As shown in FIG. 6, the anchoring system (shown in FIG. 2) allowed for the cells to be maintained for long durations during imaging. The anchoring system allowed for imaging the same, e.g., ROI, containing the same cells in the presence of various concentrations of glucose tracer.

Within the subset of cells imaged in a single field of view, a spectrum of uptake existed for each concentration of glucose tracer measured. Three examples of cells that display different relative levels of uptake (e.g., low uptake, medium uptake and high uptake) as compared to the background level of glucose tracer (5 mM) are shown in FIGS. 7A-7C. Each data set (dots) were broken into time points before uptake, during uptake and at saturation. Using a three-part heuristic model, we can fit the linear portion of the uptake and obtain the initial velocity (V₀), which were used in the Michaelis-Menten equation to determine the maximum velocity (Vₘₐₓ) of uptake for the GLUT1 receptor in RBCs when performed at various concentrations as shown in FIGS. 7D-7E.

The method disclosed in this example can provide detailed information regarding the saturation level (or plateau) of tracer within single cells as shown in the cell traces above. When this information is compiled into a histogram, either at single concentrations, or combined, as shown in FIG. 8, a characteristic distribution can be observed for a given patient. By fitting the mean and the standard deviation, information can be provided to clinician regarding a patient's glucose uptake as it can inform their HbA1c diagnostic test.

This method focuses on the relationship between glucose uptake and RBCs, which is important for better understanding diabetes. Using a microfluidic perfusion system allows the placement of RBCs and control over the solutions to which they are exposed. Using confocal imaging and analysis, the kinetics of fluorescently labeled glucose passing into and out of individual RBCs can be observed and relevant information can extracted, such as glucose influx and efflux rates, intracellular versus blood glucose levels and more. This information allows the ability to quantify the relationship between bloodstream and intracellular glucose levels to inform a more personalized diagnostic strategy for diabetes.

### Example 2: Analysis of Racial Disparity in Glucose Uptake in RBCs

This example provides further details regarding the method for determining the kinetics of glucose uptake in single RBCs described in Example 1. This example further describes the testing of this method across dozens of patients to investigate whether demographics can influence characteristic patient intracellular glucose percentage. Using the fluorescently labeled glucose analog, 2-NBDG, the results of this study indicate that patient intracellular glucose percentage, when tested at the same extracellular glucose concentration, can vary based on race (i.e., Caucasian/Hispanic vs Black). These findings offer critical insight about how RBCs vary in their glucose uptake, which is likely correlated to HbA1c formation. Because a racial disparity in diagnosis of diabetes has been previously observed, this finding adds to the discourse about why such a disparity exists. Overall, quantifying the relationship between bloodstream and intracellular glucose percentages can help inform a more personalized, and perhaps improved, diagnostic strategy for diabetes.

### Methods

Ethics. Human whole blood samples used for this study were purchased from BioIVT, LLC. Each sample purchased from BioIVT LLC were collected under an IRB protocol and certified by the IRB.

Reagents. A modified wash/homeostasis buffer was comprised of 125 mM KCl, 5 mM HEPES, 4 mM EGTA, and 5 mM MgCl₂ (to be described as KCl solution or buffer herein) (Ojelabi et al., The Journal of Biological Chemistry, 2016. 291(52): p. 26762-26772). This buffer was further modified when combined with glucose-analog tracers to be comprised of 100 mM KCl, 5 mM HEPES, 4 mM EGTA, and 5 mM MgCl₂ (i.e., 5 mM 2-NBDG). Glucose-analog tracers which were investigated were D-Glucose-Silicon rhodamine (D-glu-SiR) (Jo et al., Bioconjugate Chemistry, 2018. 29: p. 3394-3401), 6-deoxy-2-[(7-nitro-2,1,3-benzoxadiazol-4-yl)amino]-D-glucose (6-NBDG) (Barros et al., Journal of Neurochemistry, 2009. 109 Suppl 1: p. 94-100), and 2-deoxy-2-[(7-nitro-2,1,3-benzoxadiazol-4-yl)amino]-D-glucose (2-NBDG) (Yoshioka et al., Biochimica et Biophysica Acta (BBA)—General Subjects, 1996. 1289(1): p. 5-9; Yamada et al., Journal of Biological Chemistry, 2000. 275(29): p. 22278-22283; Yamada et al., Nature Protocols, 2007: p. 753-762). 2-NBDG and 6-NBDG were sourced from Invitrogen™ (Cat. No. N13195, N23106, respectively), while D-glu-SiR was synthesized in house. Calcein AM (Invitrogen™, Cat. No. C1430) was used for membrane integrity studies. GLUT1 inhibitors tested included WZB117 (EMD Millipore Corp., Cat. No. 400036-5 MG), STF-31 (EMD Millipore Corp., Cat. No. 351801-10 MG), Cytochalasin B (EMD Millipore Corp., Cat. No. C2743-200UL), Phloretin (EMD Millipore Corp., Cat. No. P7912-100 MG), and BAY-876 (EMD Millipore Corp., Cat. No. SML1774-25 MG). Dimethyl sulfoxide (DMSO) was sourced from Invitrogen (Cat. No. D12345).

Sample preparation. Human whole blood from an individual donor was drawn into sodium heparin (NaHep) and shipped cold. Samples were received directly from BioIVT LLC and placed into 4° C. refrigeration until prepared for experimentation. Samples removed from refrigeration for experimentation purposes were first inverted for three minutes and then 200 μL were drawn from the collection tubes and placed into a fresh 1.5 mL centrifuge tube containing 1 mL of 125 mM KCl solution. Samples were gently inverted 5-10× and then placed in a 4° C. centrifuge and spun using a modified RBC isolation protocol aimed at reducing clotting of whole blood and deformation of RBCs (Hanson et al., American Journal of Physiology Heart and Circulatory Physiology, 2008. 295(2): p. H786-H793). Centrifugation-based separation of RBCs was performed at 2000 RPM (490×g) for 5 minutes. After centrifugation, supernatant was removed along with buffy coat (white blood cells, etc.) using a pipette and a fresh 1 mL of KCl solution was then added. This process was repeated 3× to isolate RBCs and diminish intracellular glucose.

For glucose uptake studies, after the washing step, packed RBCs had 390 μL of KCl solution added to them along with 10 μL of 0.1 mg/mL biotinylated-α-glycophorin A+B antibodies (1:40 dilution, ABCAM plc., Cat No. ab15009). Samples were then vortexed gently and placed on a 37° C. shaker at 400 RPM for one hour for incubation. Due to the high expression of glycophorin A+B in erythrocytes, it was a simple membrane target for antibody anchoring of cells to the microfluidic imaging surface using the biotin binding Neutravidin™ system.

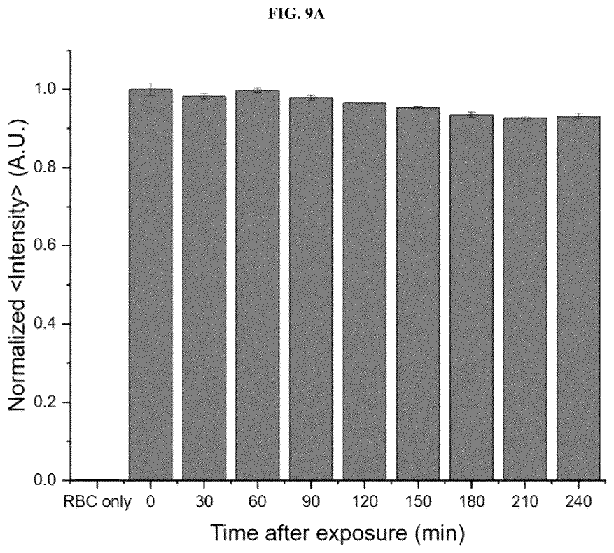

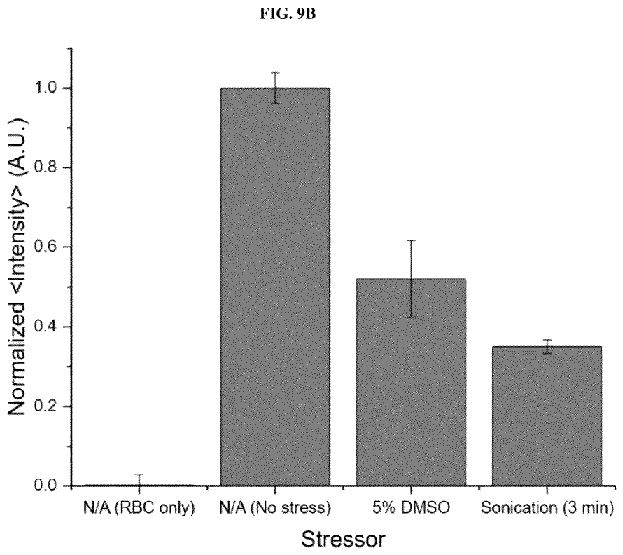

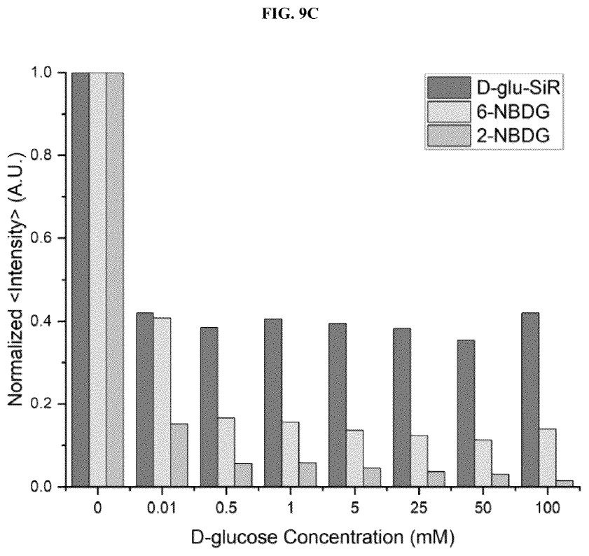

Identification of glucose-analog tracer. Human erythrocytes were isolated and washed as described in Section 2.2 but diluted to 1:100 the concentration in 125 mM KCl solution. Once washed, erythrocyte membrane integrity was then tested to confirm cell viability in the presence of the buffer system over extended periods of time (FIG. 9A). Two groups of cells were aliquoted in microcentrifuge tubes: 500 μL of cells lacking Calcein AM in 125 mM KCl solution and 500 μL of cells in 125 mM KCl solution containing 1 μM Calcein AM. The cells lacking Calcein AM were flowed through a BD FACSCanto™ flow cytometer, excited by a 488 nm laser, then signal was collected within a bandpass filter selected for FITC-A and only cells selected by forward and side scatter gating based on RBCs in KCL solution lacking Calcein AM were counted. 10,000 total events were measured per trial/sample. Subsequently, cells introduced to Calcein AM were incubated for 60 minutes, then washed one time in 125 mM KCl solution alone, and then placed into the flow cytometer using the above channel and gating. Data was normalized to the first time point of 60 minutes after introduction (FIG. 9A) which was deemed 0 minutes after incubation. A measurement was then taken every 30 minutes thereafter on the washed sample. A second control experiment showing membrane integrity effects when using Calcein AM on RBCs incubated for 60 minutes, washed then in the presence of KCl solution containing 5% DMSO or a cell solution sonicated for 3 minutes are shown in FIG. 9B. After stressors were administered, cells were immediately placed on the flow cytometer using the above conditions to attain mean FITC intensity values.

To test the tracer specificity, a competition experiment was first performed on the flow cytometer under identical settings as were used in the membrane integrity experiments above. Aliquots of solutions of RBCs containing KCl solution and 20 μM of a given glucose tracer were incubated for 60 minutes at 37° C. shaker at 400 RPM containing a selected concentration of unlabeled D-glucose (i.e., 0.00, 0.01, 0.50, 1.00, 5.00, 25.00, 50.00 or 100.00 mM). After incubation, cells were washed one time in 125 KCl solution containing the selected concentration of D-glucose and promptly measured on the flow cytometer using the conditions described above. The highest competition was observed with 2-NBDG as shown in FIG. 9C. Next, GLUT1-specific transport was further tested by preparing washed RBC solutions as described above and then incubating the RBC solutions with one of the GLUT1 inhibitors on a 37° C. shaker at 400 RPM for one hour of incubation. GLUT1 inhibitor concentrations (IC₅₀) were as follows: 100 μM phloretin (Kasahara and M. Kasahara, Biochemical Journal, 1996. 315: p. 177-182), 10 μM cytochalasin B (Kasahara and M. Kasahara, 1996), 20 μM WZB117 (Liu et al., Molecular Cancer Therapeutics, 2012. 11(8): p. 1672-1682), 10 μM STF-31 (Dedda et al., International Journal of Molecular Sciences, 2019. 20(19): p. 4962), and 10 μM BAY-876 (Dedda et al., 2019). Samples were then spun down, and supernatant was removed and fresh inhibitor/KCl solution was added and inverted. Glucose tracers were then added at working concentrations of 20 μM and incubated for 30 minutes on a 37° C. shaker at 400 RPM. After 30 minutes, cells were quickly spun down again using the protocol described in herein and supernatant was removed and replaced with fresh inhibitor/KCl solution before immediately being placed on the flow cytometer. Each sample was run on the flow cytometer using identical settings described above. It was found that 2-NBDG has the highest specificity for the GLUT1 transporter according to the examination of the panel of inhibitors tested (FIG. 1B) and the glucose competition experiments and was selected to be the glucose tracer used in the uptake measurement experiments.

Microfluidic Perfusion. An ONIX microfluidic perfusion system from CellAsic (Millipore Sigma) was used for flow-based experiments. The system comprises a perfusion pump system, which connects to a PC for multidimensional control of flow rates (converted from applied kPa, i.e., 27.6 kPA=20 μL/hr) and duration. The plates used for experiments herein are model Y04C. This plate system has a #1.5 glass bottom for imaging and contains four separate channels which have an observation region where fluid switching can occur homogenously. Plates were removed from vacuum packaging the day of experimentation and storage PBS was removed using a vacuum system. Each well was then filled with 400 μL of KCl solution and placed on the perfusion system. KCl solution was flowed through each channel and well (except for the waste well #7) at 20 μL/hr for 20 minutes to flush out any storage PBS. Next, one of the wells (#1 for each channel) was filled with 100 μL 0.1 mg/mL Pierce™ biotinylated-bovine serum albumin (B-BSA, Cat #29130, Thermo Scientific) in 125 mM KCl solution and this was then flowed through for 15 minutes total, then allowed to incubate for 15 minutes at room temperature. After the B-BSA was deposited on the glass surface of the channels, a 0.1 mg/mL solution of Neutravidin (Cat #31000, Thermo Scientific) in KCl solution was flowed for 15 minutes at room temperature to build a surface anchoring system for red blood cells.

For uptake measurements, cells incubated with biotinylated-α-glycophorin A+B antibodies were diluted 1:1 in KCl solution within well #8 and perfused using 20 μL/hr in combination with well #5 containing KCl solution only at 20 μL/hr for 10 seconds. Flowed cells were then allowed to incubate with the cell anchoring system described above for three minutes. Afterwards, well #2 containing 5 mM 2-NBDG solution was flowed for three minutes at 20 μL/hr to prime the system and wash away any non-adherent cells from the experimental observation region. KCl solution only was then perfused at 20 μL/hr for three minutes to exchange with the 2-NBDG solution. The anchored cells were first introduced to 5 mM 2-NBDG solution from well #2 by perfusing at 20 μL/hr to simulate uptake and achieving equilibrium. Immediately after this time point, well #2 was stopped, and KCl solution only from well #5 was perfused at 20 μL/hr to wash away tracer (“washout”). It should be noted that cell loading, 2-NBDG solution priming, and washout steps typically occurred with the microfluidics plate mounted to the confocal microscope stage, however, cell loading was performed using live transmission imaging. For 2-NBDG priming, the microscope was again utilized in live acquisition mode to identify the approximate center cross section of all cells in each field of view.

Confocal Microscopy. A microscope system composed of a Zeiss Axio Observer 7, a Zeiss LSM 980 scan-head, Zeiss scanning stage, Zeiss Halogen lamp 12V 100W GY6.35, 488 nm solid state laser, 40× C-Apochromat/1.2 W autocorrecting FCS M27 objective, and a PCO.Edge 4.2 BL camera were purchased from and assembled by Zeiss Microscopy (Carl Zeiss Microscopy, LLC, NY) and controlled by the Zeiss Zen software suite. For experiments that utilize the Y04C plate, the halogen illumination was used for transmission imaging to locate the imaging surface and observe cell anchoring/unbound cell washout. The halogen lamp was configured to 2.3V while the camera was configured to auto-exposure to visualize cells.

Once a central stage position on the microfluidic channel was determined visually, the position coordinates were set in the Zeiss Zen software (i.e., (0 μm, 0 μm), or (x μm, y μm) position). Four other fields of view (FOVs) were then defined relative to the center position as (−200 μm, 0 μm), (200 μm, 0 μm), (230 μm, −200 μm), and (230 μm, 200 μm) for a total of five FOVs. Stage movement to these FOVs was automated and the full wash-in/wash-out imaging protocol (see below) was run on each sequentially for a given sample. For 2-NBDG solution priming and locating approximate center cross section of cells, the live acquisition mode was used to select the objective height. Live acquisition mode reduces the acquisition time to replicate a pseudo-video rate for quick locating of cells and objective height, which is preset within the system.

For uptake measurements, the 488 nm laser was configured for excitation to a power level of 0.07% of total laser voltage (i.e., 3.5 μW before the objective) with a pixel dwell time of 0.24 μs. For detection, the detector bandwidth was set to admit wavelengths 520 nm to 560 nm with a master gain of 500 V and digital gain set to 1×. The field of view was 1944 pixels×1944 pixels (or 192.8 μm×192.8 μm) which led to a total frame time of 989.11 ms for a single acquisition. With these settings and using the 40× magnification, the pixel size was ˜0.1 μm×˜0.1 μm. Initial testing was performed using the Zeiss Experiment Designer module in the Zen software suite such that a time-series imaging sequence was automated. This sequence included 46 images with two second intervals to capture the flowing in of tracer (the “uptake” portion), followed by three sequential images during the stable, full 5 mM tracer flow (the “plateau” portion), and then 61 images with three second intervals (the “washout” portion). For the donor panel study, there was an additional automated z-stack added to the sequence after the “plateau” portion and before the “washout” portion. The z-stack consisted of 59 images taken sequentially with 0.1 μm step size. Zeiss Definite Focus was used for height verification at each step throughout the entirety of the automated imaging sequence. This sequence was repeated for each FOV. The imaging sequence was initiated sequentially with the CellAsic ONIX control software to align the two components temporally for a total elapsed experiment time of five minutes and forty seconds. For a simplified diagram depiction of the experimental preparation and imaging routine (FIG. 10).

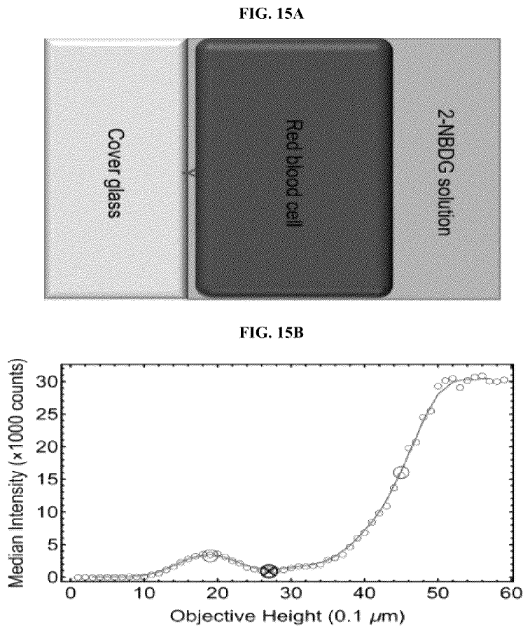

For uptake measurements to be quantitative and accurate, potential artifacts can be considered and addressed. The most significant of these is the alignment of the excitation light point spread function (PSF) with the internal cell volume and the potential contributions from extracellular fluorescence intensity. In the radial dimension, erythrocytes are large (5-8 μm) compared to the PSF (<1 μm), and careful region of interest (ROI) generation easily prevents extracellular contributions. In the axial direction, however, the heights of the PSF (˜2 μm) and erythrocytes (2-4 μm) are much more comparable. This makes the z-focus position for each individual cell of interest, and even so, thin cells may allow the tails of the PSF to excite small portions of extracellular glucose tracer above and below the cell. Given the high tracer concentration outside the cell, such a contribution is not negligible. Therefore, the measurement protocol can include axio-spatial resolution (i.e., imaging at several focal distances which is commonly referred to as a “z-stack” described above). In doing so, identification of the optimal cross section for each cell within a field of view becomes possible. The visually acquired cross-section described above serves as the central focal plane (0 μm), and a series of focal planes were imaged from −2.9 μm (below cell) to +2.9 μm (above cell) in increments of 0.1 μm. Analyzing this z-stack, series provides a “z-profile” of the median intensities for each cell (shown in FIG. 15B).

Analysis and Software. To analyze the imaging sequence described herein, a custom routine was written using Mathematica. The software can be broken into five major components: data intake and harmonization, region of interest determination for cell selection, z-stack processing for optimal cellular cross-section, calculating median pixel intensity per cell, and calculation of mean intracellular glucose. The purpose of each step will be described briefly.

Processing a single image at full, stabilized glucose tracer flow provides a high intensity background contrasted with cells which are of lower intensity and was used to define a region of interest (ROI) for each cell. Cell ROIs were eroded away from the membrane to avoid extracellular contributions and are made roughly toroidal in shape by subtracting the center section to eliminate extracellular contributions at the center of a cell due to the bi-concave nature of erythrocytes. A background ROI was defined for each FOV by dilating all cell ROIs and selecting the remaining non-cell/non-artifact region, i.e., the pure buffer with glucose tracer signal region.

Due to the typical thickness or height of an average human erythrocyte (˜2 to 2.5 μm) and the nature of a single photon excitation point-spread function (PSF), the experiment was aimed to minimize intensity contributions of the extracellular glucose tracer from the axial tails of the PSF. To this end, each cellular ROI was overlaid onto each image in a matching Z-stack group to determine the median intensity for each step in a z-stack. The intensity profile is then used to approximate the center cross-section—or optimal z-slice)—for each cell. Once the optimal z-slice for each cell is determined, the median intensity value is calculated from the cell's ROI applied to that xy-plane slice.

At this point, the median intensity for each cell from all five FOVs are tabulated and normalized to their respective background ROI. This was done to provide a metric of intracellular glucose percentage or intracellular glucose (%) defined by the following equation: Intracellular glucose (%)=median cellular ROI intensity/median background ROI intensity.

This term is used to describe the median intracellular glucose tracer percentage as compared to the median background intensity, which can be assumed to equal to 5 mM 2-NBDG (i.e., the 2-NBDG solution flowed). Once the median intracellular glucose (%) is computed for each cellular ROI, each median value can be binned into 1.0% intervals, then count the occurrences at each interval. Thus, a histogram of intracellular glucose fraction was formed for a given patient sample of erythrocytes. At this point, the mean of the histogram is defined as the mean intracellular glucose (%) for a given donor sample, and therefore analogous to the characteristic glucose uptake. This is simply termed the intracellular glucose percentage (%).

A corrected intracellular glucose percentage was also introduced to address PSF size and average cell height described above. To not exclude cells smaller than 2.1 μm due to extracellular contributions to fluorescence intensity due to the PSF extending beyond the cell, the cells were analyzed by two approaches: (i) analysis limited to cells greater than 2.1 μm in height and (ii) all cells using a correction factor. The correction factor, or height corrected intracellular glucose percentage (HCIG), for a given cell assumes the PSF is centered and uses the cell width to calculate and subtract the signal intensity contribution from the PSF tails in the extracellular solution, as follows:

$${HCIG}_{i , j} = {IF}_{i , j} - \frac{( {PSFtail}_{i , j} \times {EX}_{i , j} )}{{EX}_{i , j}} \times 100 ,$$

where IF is the mean intracellular glucose intensity signal, PSFtail is the fraction of the PSF which resides outside the cell, EF is the mean extracellular intensity signal (i.e., bulk 2-NBDG solution), i is a given cell, and j is the optimized z-profile slice for cell i. This value offers a means to approximate the correct intracellular glucose percentage despite a cell's size.

A statistical analysis was performed on data originating from a donor panel (n=45) with associated specific demographic details for racial background (Table 1). Summary statistics across all the donor samples were determined, and a t-test was used to compare the glucose uptake data by race (black or Hispanic/Caucasian) groups. The dependent variable of interest is the mean intracellular glucose (%) (for a given data grouping: all cells, height corrected and cells >2.1 μm high), and the independent variable is race. The analysis was performed using R statistical software version 1.2.1335.

TABLE 1
Summary table of patient sample information.

| | Age | | |
|---|---|---|---|
| Sample # | (yrs) | Sex | Race |
| S01 | 43 | Male | Hispanic |
| S02 | 46 | Male | Hispanic |
| S03 | 47 | Male | Hispanic |
| S04 | 19 | Male | Black |
| S05 | 49 | Male | Black |
| S06 | 54 | Female | Black |
| S07 | 25 | Female | Caucasian |
| S08 | 53 | Male | Black |
| S09 | 67 | Male | Black |
| S10 | 18 | Male | Black |
| S11 | 62 | Female | Hispanic |
| S12 | 47 | Female | Black |
| S13 | 58 | Female | Caucasian |
| S14 | 24 | Male | Black |
| S15 | 51 | Male | Black |
| S16 | 40 | Female | Black |
| S17 | 60 | Female | Hispanic |
| S18 | 44 | Female | Black |
| S19 | 19 | Male | Hispanic |
| S20 | 44 | Female | Black |
| S21 | 44 | Female | Black |
| S22 | 20 | Male | Hispanic |
| S23 | 20 | Male | Black |
| S24 | 43 | Female | Black |
| S25 | 47 | Female | Black |
| S26 | 19 | Female | Black |
| S27 | 18 | Female | Hispanic |
| S28 | 18 | Male | Hispanic |
| S29 | 62 | Female | Black |
| S30 | 21 | Male | Hispanic |
| S31 | 19 | Male | Hispanic |
| S32 | 21 | Male | Hispanic |
| S33 | 19 | Male | Hispanic |
| S34 | 19 | Male | Hispanic |
| S35 | 19 | Female | Hispanic |
| S36 | 21 | Female | Black |
| S37 | 18 | Female | Black |
| S38 | 19 | Male | Hispanic |
| S39 | 57 | Female | Caucasian |
| S40 | 66 | Female | Caucasian |
| S41 | 60 | Female | Caucasian |
| S42 | 38 | Female | Caucasian |
| S43 | 57 | Male | Caucasian |
| S44 | 21 | Male | Hispanic |
| S45 | 21 | Male | Hispanic |

Two hypotheses were defined and tested to confirm the results obtained by the team. Equal variances were assumed for the analysis. The test was performed at a 95% confidence level.

The following assumptions of the two-sample t-test were met: Independence, Normality, Homogeneity of variance, Random sampling. To check if the data followed a normal distribution, a Shapiro-Wilk statistical test was performed, and Quantile-Quantile (QQ) Plots were produced as a visual assessment of normality (not shown here). The p-values from the Shapiro-Wilk test were insignificant (p>0.05), so each set of means is concluded to follow a normal distribution. Additionally, the QQ plots were assessed and visually follow a normal distribution. The tested hypotheses were as follows: 1. Whether there is a difference in the means of the two groups (either μ1=μ2, or μ1−μ2≠0), 2. The difference of the group means is greater than zero (either μ1=μ2, or μ1−μ2>0).

### Results:

Membrane integrity and tracer specificity: This work was set out to probe patient-to-patient variability in human RBC glucose uptake to determine what, if any, demographic factors may contribute to this discordance. To this end, it was sought to probe individual RBCs for a given patient to observe (an assumed) intercellular distribution of glucose uptake. Initially, it was sought to ensure RBCs can be maintained in a homeostasis condition where glucose was omitted. The modified buffer solution was tested to ensure membrane integrity was maintained. Using Calcein AM, a unidirectional membrane penetrating dye (from extra- to intracellular), cells were allowed to incubate with the KCl solution for up to 240 minutes while periodically measuring sub-samples on a flow cytometer (FIG. 9A). After 210 minutes, less than 7.3% of the cells showed membrane deterioration (i.e., average intracellular intensity dropped below the intensity set by cells defined as in-tact by forward and side scatter gating, data not shown). As a control, membrane deterioration was forced either by incubating Calcein AM loaded cells for 3 minutes with 5% DMSO in the KCl solution or in a sonication bath. Compared to a control sample, both samples under each stressor (5% DMSO solution or 3 minutes of sonication) saw a mean decrease of 48.0% and 65.0% in intracellular Calcein AM concentration, respectively, correlating directly to membrane integrity using the flow cytometer (FIG. 9B). From these results, it was concluded that the modified buffer system could maintain cells for durations longer than needed for imaging.

Next, three fluorescent glucose-analog tracers were tested for specificity in human RBCs, two commercially available (6-NBDG and 2-NDBG) (Zou et al., J Biochem Biophys Methods, 2005. 64(3): p. 207-15) and one prepared in-house (D-glucose-SiR, as reported by Jo et al., 2018), by performing glucose competition studies and small molecule GLUT1 inhibition studies using flow cytometry. Adult human RBCs exclusively express the glucose transporter 1 (GLUT1) encoded by the SLC2A1 gene (Pragallapati and Manyam, Journal of Oral and Maxillofacial Pathology, 2019. 23(3): p. 443-449), thus the three glucose-analog tracers mentioned above were evaluated for GLUT1 specificity. In the case of competition studies, it was shown that the tracer is GLUT1 specific by performing titrations of higher and higher concentrations of unlabeled D-glucose as compared to the tracers. If the average tracer fluorescence signal is found to diminish with increasing concentrations of unlabeled D-glucose, then it means that the tracer is competing probabilistically for GLUT1 receptors to transport into cells. The glucose competition study (FIG. 9C) shows that when that the unlabeled D-glucose concentration is 5000× that of the tracers (i.e., 100 mM D-glucose), D-glucose-SIR maintained 41.9% of the mean intensity of the control sample, while 6-NBDG maintained 13.8% of the mean intensity of the control sample. 2-NBDG, on the other hand, showed a marked decrease down to 1.4% of the mean intensity of the control sample at 100 mM D-glucose. In a similar fashion, using several GLUT1 inhibitors displayed in FIG. 1B, in all but one (STF-31), 2-NBDG was consistently the most inhibited, and in the case of BAY-876 inhibitor, uptake was inhibited to 2.1% of the mean intensity of the control sample (compared to 20.6% and 28.7% for D-glucose-SIR and 6-NBDG, respectively). These tests provided significant indication that 2-NBDG was, in fact, GLUT1 specific in human RBCs. When compared to the other glucose-analog tracers, 2-NBDG was the choice for testing glucose uptake on single cells.

Perfusion flow cell: To perform single cell measurements on RBCs, a method of attaching cells was needed which would not interfere with transport of the tracer. Thus, RBCs were prepared as described, along with functionalized microfluidic channels. By making adherent cells, RBCs could be consistently exposed to 5 mM of 2-NBDG solution that can be constantly replenished within the imaging field of view (FOV) as well as control fluid exchange for 2-NBDG washout with KCl solution. Once a field of view was established, the objective height was set to approximate the center cross section of all cells present. For a given FOV, the imaging sequence was performed, and a simplified depiction of this process is shown in FIG. 11A where the sequential imaging (time-series) starts dark, and as the 5 mM tracer flows in, the intensity within the spatial intensity maps increases (shown as green pixels). Based on this contrast of intensity between intracellular regions and extracellular regions, cells can be isolated by regions of interest (ROIs) defined within a FOV. The cells within a field of view take up a fraction of the 5 mM 2-NBDG, and thus appear relatively “dark” compared to the highly fluorescent background, which is shown in FIG. 11B and FIG. 11C. This process is then reversed when the “washout” occurs where the 2-NBDG solution is exchanged with KCl solution only. Once individual cells are identified by a region of interest (ROI), and their median intensity value is calculated per frame within the time-series. To generate a simple metric which could be used to characterize intracellular glucose tracer behavior, the intracellular glucose (%) or intracellular glucose percentage was defined to be the ratio between the median fluorescent intensity inside a cellular ROI and the median (non-cell) background intensity of the entire field of view. From FIG. 11C, a steady intracellular glucose percentage was observed for each cell measured in a field of view (although only a subsample is shown).

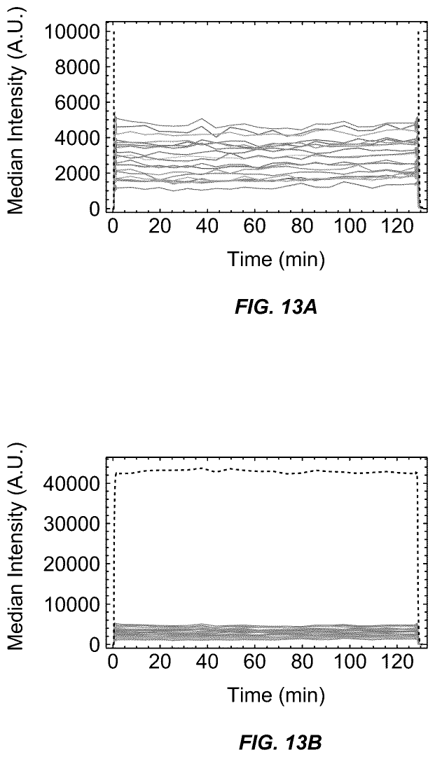

The perfusion system and fluorescent tracer, in principle, allow for the quantification of both kinetics and steady state conditions of GLUT1 transport, as demonstrated by performing a time-resolved imaging experiment (FIG. 11C) wherein 2-NBDG is first flowed into the cell compartment, maintained at steady flow for a few minutes, and then washed out again with the KCl solution. FIG. 11B shows that for any given cell (i.e., a small magnified region of FIG. 11A), the intensity of the intracellular region of erythrocytes surrounded by 2-NBDG achieve a saturation point within several seconds, which is consistent with previously recorded uptake rates for GLUT1-meditated transport. These glucose level plateaus are more clearly shown by plotting intracellular glucose (%) for a sampling of cells in FIG. 11C. This finding confirmed that any variations in glucose uptake rate (with a timescale of seconds) would not be relevant to the formation of HbA1c (with a timescale of days to weeks). Furthermore, extended duration measurements were performed to ensure the stability of the glucose level plateaus. The saturating glucose levels achieved within minutes were observed to be stable for several hours (FIGS. 13A-13B).

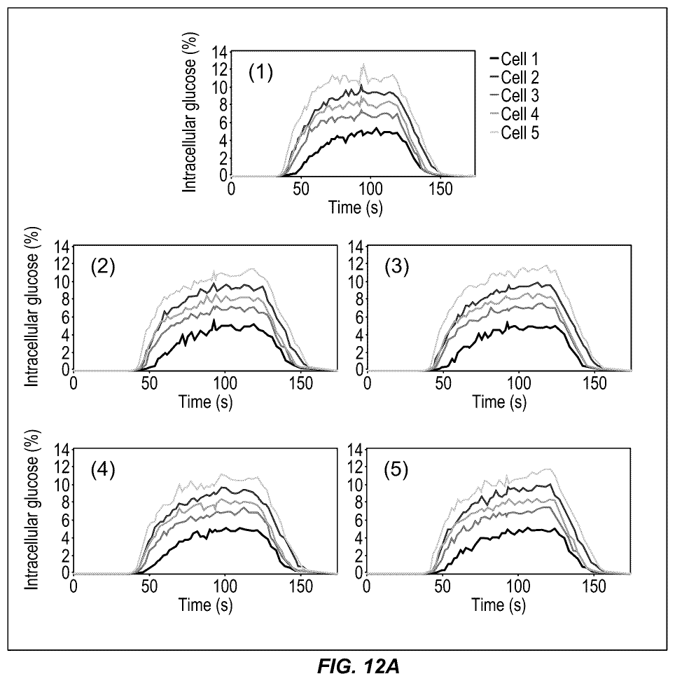

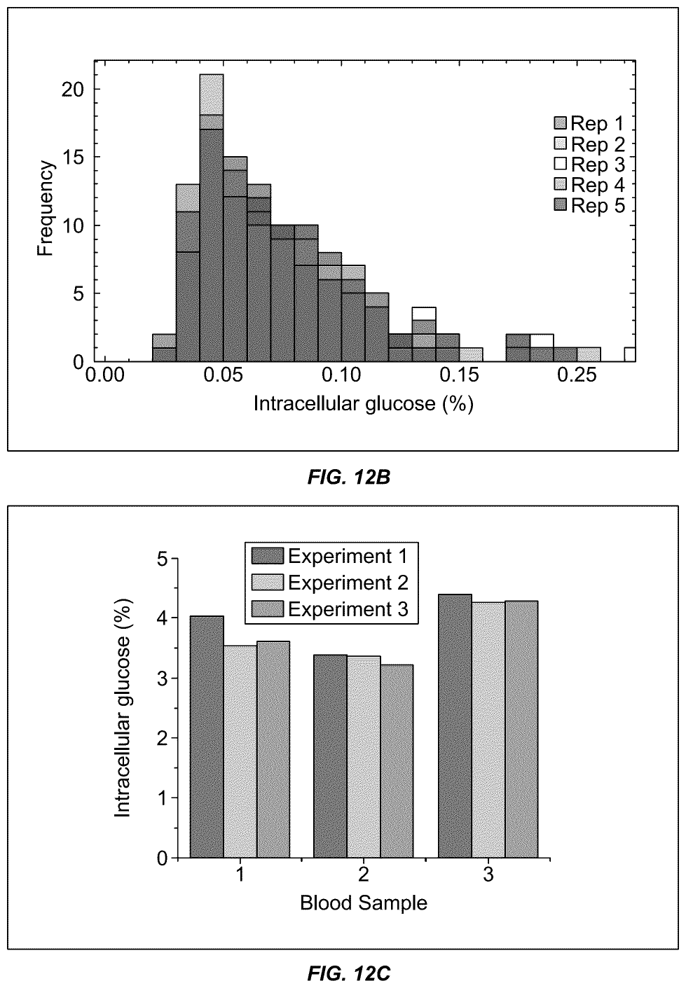

As shown in FIG. 11C, the intracellular 2-NBDG levels in each individual cell quickly reached a steady state. Unequal intracellular and extracellular glucose tracer equilibria have previously been observed, indicating a form of asymmetric GLUT1 transport or co-transport, however this is not well understood (Naftalin and De-Felice, 8.11 in Comprehensive Biophysics, E. H. Egelman, Editor. 2012, Elsevier. p. 228-264; Naftalin, Biophysical Journal, 2008. 95: p. 4300-4314; Nishimura et al., J Biol Chem, 1993. 268(12): p. 8514-8520; and Khera et al., Diabetes, 2008. 57: p. 2445-2452). This study thus focused on obtaining accurate measurements of the steady-state intracellular glucose (2-NBDG) concentrations in individual cells exposed to the continuous flow of approximately physiological glucose (˜5 mM tracer). FIG. 12A shows how individual cells exposed to 2-NBDG show a characteristic level of intracellular glucose percentage. FIG. 12A is a random sample of five cells which had the time-series experiment repeated 5 times sequentially (labeled 1-5). While minor fluctuations are observed, the overall intracellular glucose percentage at any time point for a given cell is recalled from repeat measurement to repeat measurement. In processing all ROI defined intracellular glucose percentages as measured during the saturation period (i.e., at 110s shown in FIG. 12A) within a FOV, all mean intracellular glucose percentages were counted and binned them into 1.0% intervals and formed a histogram. When looking at the histograms (FIG. 12B) of the five repeated experiments (FIG. 12A), only minor fluctuations were seen, but the overall shape of the histogram is preserved along with any features. This meant that the mean of the histogram could be used as a characteristic of a given patients intracellular glucose percentage.

Then such experiments were repeated and analysis on three separate patient blood samples. By testing the patient blood samples using this method over several days, the stability of the mean intracellular glucose percentage was tested, and the standard deviation was found to be less than 7.2% CV for all three patients tested (FIG. 12C). Lastly, the stability of the measurement in a single patient was tested over a long duration (i.e., several hours) and found that the individual cells were remarkably stable in their intracellular glucose percentage during the saturation period (FIG. 12C and FIGS. 13A-13B).

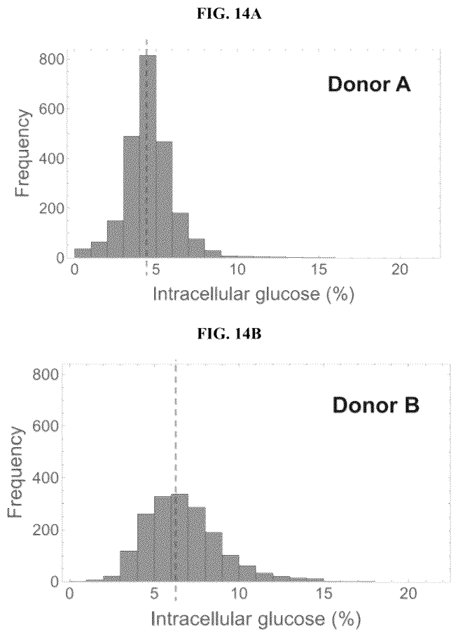

Since significant differences could be observed in intracellular glucose percentage from RBC to RBC in a single individual as shown in FIG. 11C and FIG. 12A, the characteristic mean was then sought to be the metric for testing interpatient blood sample average intracellular glucose percentage. FIGS. 14A-14B show how two patients can show significantly different histograms which lead to different mean intracellular glucose percentages. The mean of this histogram collapses much of the single cell information but offers a reliable means of measuring patient intracellular glucose levels. The two test patient blood samples were found to have a mean intracellular glucose percentage of 4.6% and 6.7% (FIG. 14A and FIG. 14B, respectively), a surprising difference of 31.3% between the means.

Z-stack analysis: During the imaging sequence, cells of different heights were observed, and because of this, there was a concern that the optimal imaging cross section for every cell within an FOV may not be captured. By measuring a z-stack, the cell height for all cells in each FOV can be approximated when 2-NBDG is at steady state perfusion (i.e., the saturation plateau shown in FIG. 11C and described in FIG. 15B). FIG. 15B shows that a typical z-profile for a cell of average height, where the x-axis represents the increment step value, and the y-axis is the median intensity for a given focal plane. The characteristic features seen in these z-profiles is first a relatively low intensity increase followed by a decrease and then a rapidly increasing intensity up to a plateau. When read in order of increasing objective height, this depicts the confocal microscope excitation point-spread function (PSF) passing from below the imaging surface (i.e., the cover slip), passing through a small layer of 2-NBDG where the cell anchor system resides, into and across the cell, then out of the cell into bulk 2-NBDG solution. It should be noted that this a small layer of 2-NBDG where the cell anchor system resides represents a diffraction limited spot between the cell and the surface of the coverslip because of the anchor system length (i.e., tens of nanometers), the center of which closely approximates the bottom of the cell. As for the higher focal planes, the top of the cell can be approximated by taking the maximum of the plateau (where the PSF is entirely in the bulk 2-NBDG solution) and locating the nearest point to half the amplitude between this maximum, and the nearest minimum to the left. This half-way point would closely approximate where the PSF is centered on the cell membrane, which is half in the bulk tracer, and half in the cell. Using these two values the height for each cell can be estimated in a field of view by locating the minimum intensity value between the identified lower and upper membranes (FIGS. 15A-15B). This is done such that the PSF is centered within the cell to minimize measured intensity contributions from the extracellular 2-NBDG solution. This z-scan protocol provides characterization of the PSF height, which enables use of a height correction to cells which may be smaller in height than the PSF.

It also may be noted that due to the size of this lower z-profile region (i.e., the leftmost “hump” in FIG. 15B), insight into the axial resolution of the excitation PSF may be gained. Several hundreds of z-profiles from numerous samples were normalized to the extracellular concentration of 2-NBDG and were fit with a simulated probability distribution function (PDF). By taking the average fitting parameters from the measured z-profiles, a PDF was calculated from these parameters and used to approximate the excitation PSF Rayleigh length, which was found to be 1.28 μm at the 1/e² distance (or 13.5% of the PSF contour excluded). However, since the cells were imaged in a 5 mM solution of 2-NBDG, 13.5% contribution from the extracellular region would vastly skew the results in favor of higher measured intracellular glucose-tracer. To account for this, the area under the curve of the simulated PDF was integrated to find where 99.0% of the PSF is within the cell. This provided an estimated PSF height of 2.1 μm, which when compared with the calculated minimum average cell height of 2.4 μm, there was confidence that several of the measured erythrocytes are below the 2.1 μm threshold.

By implementing a firm threshold on cell height at 2.1 μm, cells which fell below this value were excluded from further analysis due to contributions from extracellular florescent buffer, which can lead to greater than 10% error in the intracellular 2-NBDG measurements. By repeating this z-stack imaging and analysis protocol for five total fields of view for a given patient sample, the assay was ready to perform intracellular glucose fraction analysis on enough cells. Purposefully, it is recalled that FIGS. 11A-11B which show how histograms inclusive of only cells whose height is greater than 2.1 μm maintain differences in their distributions (as shown in FIGS. 14A-14B).

Patient panel data: Since the percent difference between the samples was greater than 10% error (as noted above), this prompted the investigation of a patient panel study focused on demographic differences among patients. With other investigations showing that differences in patient HbA1c to their fasting glucose levels is correlated with race and age (Cheng et al., JAMA, 2019. 322(24): p. 2389-2398), this panel would follow a regime as displayed in Table 2. These conditions are set to explore the most sizable discordances observed between Caucasian male patients younger than 21 and Black female patients older than 50. For each donor sample (n=20 with Black ancestry and n=25 with Caucasian or Hispanic ancestry), five fields of view were acquired with a 40× water objective providing a total of 1000 to 3400 RBCs measured per individual. The optimal intracellular focal plane was determined for each RBC to accurately assess median intracellular fluorescence, and when compared as a ratio with the extracellular medium offers a quantifiable metric. This characteristic intracellular glucose fraction for each individual was normalized to the mean value across all measured individuals, and the normalized fractions were compared across the two racial groups.

TABLE 2
Patient panel criteria breakdown and corresponding number of patients per category.

| Race: | Caucasian/Hispanic | | | | Black | | | |
|---|---|---|---|---|---|---|---|---|
| Sex: | M | M | F | F | M | M | F | F |
| Age (yrs): | under 25 | over 38 | under 25 | over 38 | under 25 | over 38 | under 25 | over 38 |
| # of Samples: | 11 | 4 | 3 | 7 | 4 | 4 | 3 | 9 |

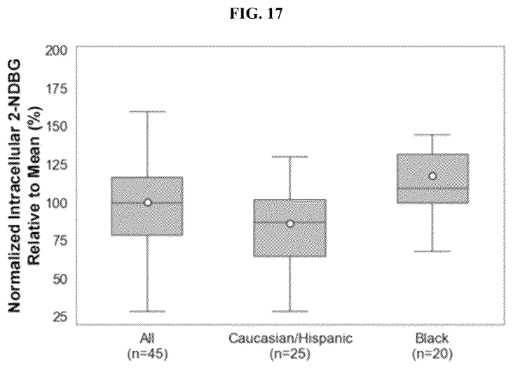

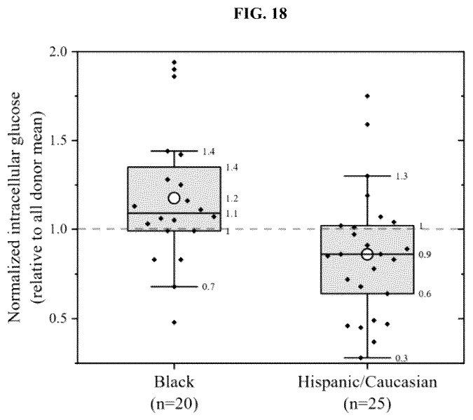

The analysis of the patient panel showed an observed difference in mean glucose percentage between race categories (FIG. 16A) when evaluating RBCs which have been cell height corrected as discussed above. The intracellular glucose percentage for Black and Caucasian/Hispanic patients was found to be 0.057±0.018 and 0.044±0.018 (mean±1 SD), respectively. However, to negate any potential bias of removing cells of smaller size than the 2.1 μm threshold from analysis, a correction factor was implemented for all cells in the panel study. Thus, FIG. 16B shows how when all cells are included with corrected intracellular glucose percentage (HCIG), the means slightly change (0.060±0.020 for Black patients, and 0.044±0.018 for Caucasian/Hispanic patient), but the differences between race remain. FIG. 16C and FIG. 16D show the results of two sample t-tests performed for cells filtered by height ≥2.1 μm, and all cells with CIGP, respectively. For both analyses, the p-value for both hypothesis tests was found to be significant (p<0.05), which leads to rejection of the null hypotheses and conclude that there is a significant difference in the mean intracellular glucose fraction of red blood cells between Black and Caucasian/Hispanic groups. This statistical analysis shows that the difference in the mean intracellular glucose fraction between the two groups is greater than zero. As shown in FIG. 17, those with Black ancestry had higher mean (+/−SD) uptake (118%+/−39%) than those with White ancestry (86%+/−36%). Similarly, FIG. 18 depicts the mean intracellular glucose percentage data as normalized to the mean of all donor samples measured (or the all donor mean). As shown in FIG. 17 and FIG. 18, a clear delineation can be drawn from the all donor mean and the two racial sub-groups, as the Black sub-group mean normalized intracellular glucose is 1.18, while the Hispanic/Caucasian sub-group mean normalized intracellular glucose is 0.86. This corresponds to a +16.51% difference from the all donor mean for Black donors, and a −15.05% difference from the all donor mean for Hispanic/Caucasian donors. Between the two sub-groups, there is an overall 31.37% difference between their mean normalized intracellular glucose. Therefore, race has been observed to be a factor which contributes to differences in measured intracellular glucose fractions.

### Discussion

The percentage of glycated hemoglobin fraction—or HbA1c—has been the cornerstone for diagnosis and treatment of diabetes mellitus for decades as it represents a stable, time-averaging (˜3 months) of the fluctuations of glucose levels within the bloodstream. More recently, the development of continuous glucose monitors (CGMs) has offered another, and potentially more direct, metric for blood glucose, and their proliferation makes it possible to directly compare the actual time-averaged CGM data with HbA1c on a given individual. The relationship between CGM and HbA1c results is not always consistent and can differ from patient to patient. Analysis of large clinical data sets has shown that these differences correlate with age, race, and sex, suggesting underlying biochemical variations.

However, it has been suggested that there is a large variation in the relationship between average glucose levels and HbA1c, creating the need to understand glucose variability at the cellular level. Due to the wide variation of average glucose levels that an HbA1c value can represent, it was investigated whether glucose uptake in RBCs was different when exposed to identical physiological levels of extracellular glucose (Perlman et al., Diabetes Technology & Therapeutics, 2021. 23(4): p. 253-258). This method offered a path towards reproducible experimentation on single RBCs, which could then be tested across several patients. For the first time, this example demonstrates that intracellular/extracellular glucose percentages can be measured at the single cell level using a fluorescently labeled glucose analog. Further, as discussed in more detail herein, a donor panel study indicates that the characteristic intracellular glucose percentages statistically differ based on race (i.e., Caucasian/Hispanic vs Black), and that RBC intracellular glucose levels show significant variability both from cell-to-cell and from donor-to-donor, which in turn impacts HbA1c formation.

The findings using this method reveal a wide variation in intracellular glucose levels, with a mean coefficient of variation (%) of all tested samples of 41.8% (5.6% mean and 2.3% mean standard deviation). Subgroup analysis by demographic factors (i.e., race, age, sex), found statistically significant difference between race and intracellular glucose fraction. A 26.9% difference was observed between the means of intracellular glucose percentage of Blacks and Hispanics/Caucasians, and within each subgroup CV % of 32.9% and 41.7% was observed, respectively. Therefore, it appears that the findings of observable differences in intracellular glucose equilibrium fraction correlate, at least in some degree, to the effect race seemingly has upon HbA1c values when all tested patients are at the same fasting blood glucose level (Karter et al., Diabetes Technology & Therapeutics, 2023. 25(10); Cheng et al., 2019; and Bergenstal et al., Annals of Internal Medicine, 2017. 167(2): p. 95-102).

On the method itself, in pursuit of investigating glucose uptake in human RBCs, the presently disclosed method enables researchers to monitor intracellular glucose fractions at the single cell level. This method provides an accessible means to investigate individual variation in glucose uptake in RBCs, and longitudinal changes within the same person. This approach was developed with commonly available lab instrumentation. Due to this, there is potential to investigate and quantify other forms of membrane transport (or ligand internalization) than just glucose transport. Thus, the capabilities may extend beyond cytoplasmic equilibration of internalized ligand, to the study of membrane transport receptor kinetics. While not analyzed directly here, transport kinetics can be a useful application of the presented method.

Using confocal microscopy can have limitations, two of which were expressly seen during this study. At 5 mM 2-NBDG, bulk solution was penetrated without any observable inner filter effects, the phenomenon of absorption of excitation or emission light by the sample which can reduce the measured fluorescence intensity. However, at very high tracer concentrations (i.e., 30 mM 2-NBDG not shown here) reductions in fluorescence signal were observed as a function of focal depth. This should be tested when setting up protocols on a different instrument or when using a different fluorescent ligand. Second, the relative heights of the excitation PSF and the cells needs careful attention to avoid artifacts. Excitation light from the tails of the PSF originating outside the cells can bias intracellular measurement results, and thus good estimates of these dimensions can be determined prior to performing live-cell imaging. For a PSF and cells of similar height, the correction factor method can be applied to any cell line.

Other limitations of the method stem from the choice of tracer. While it was found that 2-NBDG is GLUT1 specific in human erythrocytes, others have found reduced specificity in other cell lines (Hamilton et al., Biochimie, 2021. 190: p. 1-11). 2-NBDG, as a fluorescent glucose analog, is a little less than twice the molecular weight of D-glucose (342 vs. 180 g/mol). Different intracellular glucose tracer fractions have been observed in other cell lines using other tracers, which indicates that physical differences between D-glucose and glucose tracers (e.g., 2-NBDG) may affect absolute transport rates and transporter binding affinities. Nevertheless, if conditions are identical for all experiments, then the differences observed from sample-to-sample and cell-to-cell should correlate with the differences in physiological glucose transport. Likewise, cell membrane integrity should be examined prior to all studies to verify stability for long duration imaging. In human erythrocytes, long duration viability through sustained membrane (FIG. 9) and prolonged exposure to physiological levels of the glucose analog (2-NBDG) with sustained intracellular fractions integrity was observed (FIG. 13). While 2-NBDG has been shown to remain in cells when converted to 2-NBDG 6-phosphate in E. coli, and ultimately decomposed into a non-fluorescent form, the rate of decomposition is likely overwhelmed by the rate of the transport mechanism (Yoshioka et al., Bioscience, Biotechnology, and Biochemistry, 1996. 60(11): p. 1899-1901). Therefore, while it is unknown whether this metabolite occurs in human erythrocytes, it may be irrelevant in comparison to GLUT1 transport rates and a constant renewal of extracellular 2-NBDG observed during flow-based experimentation (Nishimura et al., 1993). It is believed that this means that the dynamic equilibrium observed within the intracellular region of erythrocytes of 2-NBDG is likely correlated with transport as opposed to decomposition.

Throughput and statistics may be improved if kinetics is not the goal of a study using this method. It is advised that if kinetics is not required, then steady state uptake measurements are possible in non-microfluidic chambers. This means that the z-stack protocol described above can be implemented automatically across numerous fields of view to improve statistics (motorized stage required). Meanwhile, the correction factor implemented in analysis will allow recapturing of all cell statistics and removal any potential bias in the study due to cell size. By focusing on commercially available instrumentation and tracers, a simplified method has been described with the ability to be widely adopted. Furthermore, analysis suggestions and corrections are provided which will aid in more accurate measurements of membrane transport. This method is likely versatile across numerous cell lines and highly modifiable depending on one's desired investigation.

In particular, the presently disclosed method offered a path towards reproducible experimentation on single RBCs, which could then be tested across several donors. The findings reveal a wide variation in intracellular glucose levels, with a mean coefficient of variation (%) of all tested samples of 39.9%(5.1% mean and 2.0% standard deviation). A statistically significant 30.8% difference was observed between the means of Black and Hispanic/Caucasian intracellular glucose percentages, and within each subgroup a CV % of 32.9% and 41.7%, respectively, was observed. One donor with a tendency toward higher intracellular glucose concentration than another donor would increase the probability of glucose-hemoglobin interactions, thus yielding a higher HbA1c percentage. This explanation and these results align with clinically observed differences in HbA1c values when all tested donors are at the same fasting blood glucose level. Therefore, the data presented herein offers quantitative evidence explaining the biological mechanism underlying the discordance, which is rooted in donor-to-donor variability, and helps set the stage for personalized flexibility in diabetes diagnostic criteria. It is anticipated that this method, and the observations made by implementing this method, will be of broad interest to the membrane study community and the diabetes-community alike, with implications towards personalized medicine.

Although the presently disclosed subject matter and its advantages have been described in detail, it should be understood that various changes, substitutions and alterations can be made herein without departing from the spirit and scope of the present disclosure. Moreover, the scope of the present application is not intended to be limited to the particular embodiments of the process, machine, manufacture, and composition of matter, means, methods and steps described in the specification. Accordingly, the appended claims are intended to comprise within their scope such processes, machines, manufacture, compositions of matter, means, methods or steps.

Various patents, patent applications, publications, product descriptions and protocols are cited throughout this application, the disclosure of which are incorporated herein by reference in their entireties for all purposes.

1. A method for analyzing glucose internalization in single red blood cells, comprising:
(a) anchoring a population of red blood cells to a substrate to generate a population of anchored red blood cells;
(b) contacting the population of anchored red blood cells with a fluorescently labeled glucose analog;
(c) obtaining sequential images of the population of anchored red blood cells or a subset thereof of (b) for a length of time; and
(d) analyzing the internalization of the fluorescently labeled glucose analog in one or more single red blood cells of the population of anchored red blood cells or the subset thereof.

2. A method for analyzing glucose internalization in single red blood cells, comprising:
(a) providing a population of red blood cells;
(b) anchoring the population of red blood cells to a substrate to generate a population of anchored red blood cells;
(c) contacting the population of anchored red blood cells with a fluorescently labeled glucose analog;
(d) obtaining sequential images of the population of anchored red blood cells or a subset thereof of (c) for a length of time; and
(e) analyzing the internalization of the fluorescently labeled glucose analog in one or more single red blood cells of the population of anchored red blood cells or the subset thereof.

3. The method of claim 1, wherein the population of red blood cells is obtained from a single patient.

4. (canceled)

5. The method of claim 3, wherein the patient is nondiabetic, has prediabetes or has diabetes.

6. The method of claim 1, wherein the fluorescently labeled glucose analog is 2-NBDG.

7. The method of claim 1, wherein the fluorescently labeled glucose analog is contacted with the population of anchored red blood cells at a concentration from about 1 mM to about 30 mM.

8. The method of claim 1, wherein the population of anchored red blood cells are contacted with the fluorescently labeled glucose analog at more than one concentration, at more than two concentrations, at more than three concentrations, at more than four concentrations or at more than five concentrations.

9. The method of claim 1, wherein anchoring the population of red blood cells to the substrate comprises:
(i) coating the substrate with a serum albumin-biotin conjugate coupled to an avidin or an analog thereof to generate a coated substrate;
(ii) contacting the population of red blood cells with an antigen-binding molecule-biotin conjugate to generate a population of red blood cells coupled to the antigen-binding molecule-biotin conjugate; and
(iii) contacting the population of red blood cells coupled to the antigen-binding molecule-biotin conjugate with the coated substrate of (i) to generate a population of anchored red blood cells.

10. The method of claim 9, wherein the antigen-binding molecule-biotin conjugate binds to a protein on the surface of the red blood cells, wherein the protein is not a GLUT1 receptor.

11. The method of claim 10, wherein the protein on the surface of the red blood cells is a glycophorin.

12. The method of claim 1, wherein the population of red blood cells comprises from about 5 to about 10,000 red blood cells.

13. The method of claim 1, wherein (e) analyzing the internalization of the fluorescently labeled glucose analog in each single red blood cell of the population of anchored red blood cells or the subset thereof comprises (i) determining an intracellular level of the glucose analog in each red blood cell, (ii) determining a Vₘₐₓ for each red blood cell and/or (iii) determining a ratio of the intracellular level to the extracellular level of the glucose analog of each red blood cell.

14. The method of claim 13 further comprising (i) determining an average or median intracellular level of the glucose analog in the population of red blood cells or the subset thereof, (ii) determining an average or median Vₘₐₓ of the population of red blood cells or the subset thereof and/or (iii) determining an average or median ratio of the intracellular level to the extracellular level of the glucose analog of the population of red blood cells or the subset thereof.

15. (canceled)

16. (canceled)

17. (canceled)

18. (canceled)

19. The method of claim 13 further comprising:
(i) comparing the intracellular level of the glucose analog in each red blood cell or the average or median intracellular level of the glucose analog to a reference intracellular level;
(ii) comparing the Vₘₐₓ or the average or median Vₘₐₓ to a reference Vₘₐₓ; and/or
(iii) comparing the ratio or the average or median ratio to a reference ratio.

20. (canceled)

21. (canceled)

22. The method of claim 19, wherein the reference intracellular level, the reference Vₘₐₓ and/or the reference ratio are values obtained from a plurality of subjects having at least one demographic metric in common with the patient.

23. A method of providing a personalized diagnosis and/or treatment to a patient using the intracellular level of the glucose analog, the Vₘₐₓ and/or the ratio of the intracellular level to the extracellular level of the glucose analog of claim 13.

24. A method of providing a personalized diagnosis and/or treatment to a patient using the average or median intracellular level of the glucose analog, the average or median Vₘₐₓ and/or the average or median ratio of the intracellular level to the extracellular level of the glucose analog of claim 14.

25. (canceled)

26. A method of predicting, modifying, evaluating, correcting and/or adjusting a patient's HbA1c using the Vₘₐₓ of claim 13 or an average or median Vₘₐₓ.

27. A kit for performing the method of claim 1.

28. A system for performing the method of claim 1.
